(19) United States
(12) Patent Application Publication
(10) Pub. No.: US 2025/0152021 A1
(43) Pub. Date: May 15, 2025

(54) Wearable Optical Device For Health Monitoring

(71) Applicant: Washington University, St. Louis, MO (US)

(72) Inventors: Christine O'Brien, St. Louis, MO (US); Victor Davila-Roman, St. Louis, MO (US)

(21) Appl. No.: 18/948,364

(22) Filed: Nov. 14, 2024

Related U.S. Application Data
(60) Provisional application No. 63/598,560, filed on Nov. 14, 2023.

(51) Int. Cl. A61B 5/02; A61B 5/024; A61B 5/026

(52) U.S. Cl. CPC A61B 5/02028; A61B 5/02444; A61B 5/0261

(57) ABSTRACT

Systems and methods are described herein for monitoring hemodynamic status, including: operating a light source to emit light toward a portion of a subject; obtaining subject data via the imaging device, the subject data including at least hemodynamic parameters of the subject; analyzing the subject data to determine if a parameter of the hemodynamic parameters is indicative of a health issue relating to blood flow; and generating an output regarding the health issue.

### CROSS-REFERENCE TO RELATED APPLICATION

This application claims priority to U.S. provisional application No. 63/598,560 filed on Nov. 14, 2023, the entire content and disclosures of which are incorporated herein by reference in their entireties.

### STATEMENT REGARDING FEDERALLY SPONSORED RESEARCH & DEVELOPMENT

This invention was made with government support under HD103954 awarded by the National Institutes of Health. The government has certain rights in the invention.br

### FIELD

The field of this disclosure relates generally to optical devices for health monitoring, and more specifically to systems, devices, and methods for non-invasively measuring peripheral hemodynamics in a subject, including a wearable optical device for health monitoring.

### BACKGROUND

Postpartum hemorrhage (PPH), defined as the excessive loss (e.g., 1 L or more) of blood within 24 hours after birth (e.g., following delivery), is the leading cause of maternal mortality worldwide with an estimated 14 million cases each year resulting in 130,000 deaths. Importantly, PPH has been noted as the most preventable cause of maternal mortality. The leading factors causing preventable PPH are delays in diagnosis and treatment. PPH prevention is especially critical in low resource settings that often have low blood stores for transfusion and consequently rely primarily on early pharmacologic treatment for PPH. The United States (US) has the highest maternal mortality rate of any developed country, where the most commonly used method for PPH diagnosis is a visual estimation of blood loss, a method known to underestimate blood loss. PPH is more prevalent in low- and middle-income countries and still accounts for ˜11% of maternal deaths in the US, and >90% of deaths are preventable. Outcomes of PPH are highly dependent on swift diagnosis and treatment initiation and thus early diagnosis of PPH is of paramount importance. There is a need for an early and accurate PPH detection and/or alert system and treatment initiation.

Preeclampsia (PE) is another leading cause (e.g., 3^{rd} leading cause) of maternal mortality worldwide. PE is a gestational disease affecting 2-10% of pregnancies globally, and may present clinically as hypertension and proteinuria. PE morbidity and mortality disproportionately affects low-resource communities. Early diagnosis and treatment is paramount, however, limitations to current clinical methods make PE diagnosis difficult. For example, most PE symptoms are (i) not specific, (ii) represent late-state-disease, and/or (iii) require laboratory evaluation. Moreover, diagnostic tools require high-level resources. For example, serum-based and urine-based diagnostics requires consumables and high-level lab facilities, and blood pressure monitors alone cannot diagnose PE. There is a need for an early and accurate PE detection and/or alert system.

PE may be characterized by endothelial dysfunction (ED), a cardiovascular disorder. Flow mediated dilation (FMD) is the body's mechanism to reduce vascular resistance in response to increased cardiovascular stress, and ED is defined as an impairment in this mechanism. Vasodilation may be induced by nitrous oxide (NO). Current diagnostic methods include brachial arterial reflex testing (BART), and using an EndoPAT 2000 device for testing of arterial health/endothelial vasodilator function. There is a need for an early and accurate PE detection and/or alert system.

Monitoring peripheral hemodynamics may be useful for detection of PPH, PE, and/or other blood flow and blood loss conditions. Known devices for monitoring peripheral blood flow, however, typically provide relative measurements that make clinically meaningful interpretation difficult, especially when a “healthy” baseline cannot be obtained. Moreover, many such devices are affected by skin color, potentially resulting in different measurements under identical physiological conditions depending on the skin color of the patient.

What is needed are systems, methods, and devices for (i) non-invasive detection of cardiovascular health indicators, capable of early and accurate detection and/or alerting of PPH, PE, related disorders such as ED, and/or other blood flow and blood loss conditions, (ii) early treatment initiation, and/or (iii) providing subject-specific perfusion ranges that can be determined regardless of whether a healthy baseline measurement can be acquired.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION OF THE DISCLOSURE

One aspect of the present disclosure is a computing device for monitoring hemodynamic status, the computer device including at least one processor in communication with at least one memory device, a light source, and an imaging device, the at least one processor programmed to: operate the light source to emit light toward a portion of a subject; obtain subject data via the imaging device, the subject data including at least hemodynamic parameters of the subject; analyze the subject data to determine if a parameter of the hemodynamic parameters is indicative of a health issue relating to blood flow; and generate an output regarding the health issue, the output configured to apprise a user of the health issue.

Another aspect of the present disclosure is computer-implemented method for monitoring hemodynamic status, the method implemented by a computer device including at least one processor in communication with at least one memory device, a light source, and an imaging device, the method including: operating the light source to emit light toward a portion of a subject; obtaining subject data via the imaging device, the subject data including at least hemodynamic parameters of the subject; analyzing the subject data to determine if a parameter of the hemodynamic parameters is indicative of a health issue relating to blood flow; and generating an output regarding the health issue, the output configured to apprise a user of the health issue.

Yet another aspect of the present disclosure is one or more non-transitory computer-readable storage media for a computing device providing monitoring of hemodynamic status, the one or more non-transitory computer-readable storage media comprising a plurality of instructions stored thereon that, in response to being executed, cause the computing device to: operate the light source to emit light toward a portion of a subject; obtain subject data via the imaging device, the subject data including at least hemodynamic parameters of the subject; analyze the subject data to determine if a parameter of the hemodynamic parameters is indicative of a health issue relating to blood flow; and generate an output regarding the health issue, the output configured to apprise a user of the health issue.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments described herein may be better understood by referring to the following description in conjunction with the accompanying drawings. Those of skill in the art will understand that the drawings, described below, are for illustrative purposes only. The drawings are not intended to limit the scope of the present teachings in any way.

FIG. 1A illustrates a laser speckle imaging (LSI) system according to one embodiment of the present disclosure.

FIG. 1B illustrates aspects of a laser speckle device of the LSI system according to FIG. 1A.

FIG. 1C illustrates an embodiment of one configuration of a laser speckle device according to one embodiment of the present disclosure.

FIG. 1D illustrates another embodiment of a laser speckle device according to one embodiment of the present disclosure.

FIG. 1E illustrates an alternative configuration of a laser speckle device according to one embodiment of the present disclosure.

FIG. 1F illustrates a real-world depiction of a laser speckle device according to one embodiment of the present disclosure.

FIGS. 2A-2D illustrate SPG downsampling and FFT results obtained from a laser speckle device according to one embodiment of the present disclosure (FIG. 2A—full size, FIG. 2B—half size, and FIG. 2C—quarter size).

FIGS. 3A-3C illustrates aspects of PPH (FIG. 3A—PPH in one subject, FIG. 3B—PPH in another subject, FIG. 3C—stages of PPH).

FIGS. 4A and 4B illustrate aspects of blood loss compensation (FIG. 4A is a diagram illustrating peripheral vasoconstriction, FIG. 4B is a diagram illustrating peripheral vasoconstriction in connection with a laser speckle device according to one embodiment of the present disclosure).

FIGS. 5A and 5B illustrate aspects of laser speckle contrast imaging according to one embodiment of the present disclosure (FIG. 5A is a diagram illustrating laser speckle contrast imaging (LSCI), FIG. 5B is a diagram illustrating aspects of LSCI and PPH detection).

FIGS. 6A and 6B illustrate aspects of LSFI signals (FIG. 6A is a plot 600 illustrating pulsatile signal, FIG. 6B is a plot 602 illustrating signal mean).

FIGS. 7A and 7B illustrate aspects of a flow phantom experimental setup (FIG. 7A illustrates a configuration of an embodiment of a flow phantom experimental setup, FIG. 7B illustrates a diagram of flow phantom results).

FIGS. 8A-8P illustrate aspects of a swine hemorrhage model (FIG. 8A illustrates a setup for testing a swine, FIG. 8B is a plot illustrating vein collapse, FIGS. 8C-8H illustrates swine hemorrhage model results for 6 swine, FIGS. 8I-8L illustrate plots regarding quantitative analysis of blood loss, FIGS. 8M-8P illustrate plots regarding quantitative analysis of resuscitation).

FIGS. 9A to 9D illustrate aspects of testing of healthy human volunteers (FIG. 9A—heathy human volunteer protocol, FIG. 9B—two subject groups, FIG. 9C—Cesarean delivery results—stable medication levels, FIG. 9D—Cesarean delivery results—dynamic medication levels).

FIGS. 10A and 10B illustrate PE aspects.

FIGS. 11A-11C illustrate aspects of light-based waveforms (FIG. 11A illustrates light-based cardiac cycle waveforms for providing diagnostic features, FIGS. 11B and 11C illustrate how light-based SPG waveforms overcome limitations of PPG).

FIGS. 12A and 12B illustrate results from swine studies.

FIG. 13 illustrates results from a cardiac catheterization lab.

FIG. 14 illustrates light-based detection of ED.

FIGS. 15A and 15B illustrate results of an embodiment of a research method for FMD comparisons to determine ED (FIG. 15A illustrates FMD raw waveform, FIG. 15B illustrates FMD mean SPG waveform).

FIGS. 16A-16E illustrate additional swine test results.

FIGS. 17A-17G illustrate comparisons and tests for detecting ED (FIG. 17A illustrates the vasodilation mechanism, FIG. 17B illustrates a BART/EndoPAT 2000 diagnostic setup, FIC. 17C illustrates results of the workflow of the FIG. 17B setup, FIG. 17D illustrates Raw LSFI signal during BART study—observed an increase in LSFI during ischemia recovery and decrease during ischemia, FIG. 17E illustrates mean LSFI signal during BART study, FIG. 17F illustrates EndoPAT2000 results during BART study, FIG. 17G illustrates all three methods come to the same conclusion showing healthy endothelium.

FIG. 18 illustrates a schematic of an embodiment of a system shown in FIG. 1A.

FIG. 19 illustrates a schematic of an embodiment of a computing device shown in FIG. 1A.

FIG. 20 illustrates an example configuration of a laser speckle device

FIG. 21 illustrates an example process flow according to one embodiment of the present disclosure.

Corresponding reference characters indicate corresponding parts throughout the drawings. There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

The following detailed description illustrates embodiments of the present disclosure by way of example and not by way of limitation. The description enables one skilled in the art to make and use the disclosure, describes several embodiments, adaptations, variations, alternatives, and uses of the disclosure, including what is presently believed to be the best mode of carrying out the disclosure. The disclosure is described as applied to an example embodiment, namely, methods and systems for (i), (ii).

In various aspects, systems, devices, and methods for non-invasively monitoring peripheral hemodynamics in a subject are disclosed herein, including arterial health and cardiovascular disorders. More particularly, this disclosure relates to normalization and calibration for such systems, devices, and methods. Various aspects may include monitoring a subject for (i) hemorrhage, including, but not limited to post-partum hemorrhage (PPH), (ii) PE, (iii) ED, and/or (iv) or other blood flow and/or blood/vascular disorders.

During hemorrhage, two important compensatory mechanisms occur: 1) blood is shunted from the periphery to vital organs by constricting peripheral vessels; 2) interstitial fluid is transferred into vessels to maintain blood volume, effectively reducing hemoglobin (Hb) concentration and hematocrit (Hct). These compensatory mechanisms help stabilize the patient and delay the time until global vascular indicators such as blood pressure and heart rate are affected. Thus, the monitoring of peripheral blood flow and blood content can detect relatively minor decreases in Hb and Hct that serve as early indicators of hemorrhage.

Optical technologies are well-suited to noninvasively measure blood flow and blood content. Laser speckle imaging directly measures flowing blood cells and the laser speckle flow index (LSFI) is proportional to velocity. Optical spectroscopy provides quantification of blood and tissue oxygenation (near-infrared region), as well as quantification of water (infrared region), enabling observation of water transfer to the vasculature during hemorrhage. Optical monitoring techniques are non-ionizing, label-free, fast, and can be implemented using small and wearable devices to provide a continuous ergonomic sensing system. Preliminary experiments described in the Examples below demonstrate sensitivity to reduced perfusion in vivo using laser speckle imaging, and/or optical spectroscopy measures significant differences between blood samples diluted with saline to physiologic levels seen in PPH.

Optical monitoring of blood flow and blood content has numerous advantages: sensitivity to multiple intrinsic biological chromophores (melanin, deoxy- and oxyhemoglobin, lipids, proteins, and water) depending upon the optical wavelengths used; ability to detect and quantify blood flow; high potential for small, simple, and wearable hardware; and rapid results. Such characteristics are ideal for patient monitoring, as evidenced by the pulse oximeter, an optical device used globally for patient monitoring. Optical spectroscopy-based tools have been developed for in vitro and in vivo measurement of hemoglobin, and continuous noninvasive optical spectroscopy tools have been used extensively in critical care patients to monitor changes in Hb concentration caused by hypovolemia. Although the perfusion index is known to be skewed and has high patient variability, previous results are encouraging and show that non-invasive optical measures can detect early signs of postpartum blood loss.

In various aspects, a multifunctional sensing system to track Hb concentration and peripheral perfusion for the monitoring and/or early detection of postpartum hemorrhage (PPH) is disclosed. The disclosed system may include an LSFI (laser speckle flow index) sensor and/or a multispectral Hb sensor, where the sensor may include a laser and a camera alone or in combination. In various aspects, the disclosed system synergistically combines laser speckle imaging for blood perfusion measurements and near infrared/short wave infrared (NIR/SWIR) spectroscopy for monitoring Hb, to provide tracking of the two separate and independent compensatory mechanisms of PPH. Other embodiments include only the laser speckle imaging or only the NIR/SWIR spectroscopy.

The LSFI (laser speckle flow index) sensor uses laser speckle imaging of peripheral skin and muscle tissues to monitor peripheral perfusion. The laser speckle sensor performs peripheral perfusion monitoring which is proportional to blood flow velocity to provide a more direct measure of perfusion than the perfusion index. In one aspect, the LSFI sensor includes a 785 nm laser diode and a video camera to obtain laser speckle contrast images. The laser speckle contrast images are processed using established algorithms to obtain laser speckle flow index images indicative of peripheral perfusion. In some aspects, the LSFI sensor is a wearable LSFI (laser speckle flow index) sensor positioned over a muscle and gently held in place with a band, ensuring placement over muscles in order to measure both skin and muscle blood flow.

In various aspects, data obtained using the disclosed laser speckle device are transferred to secure cloud storage using a wireless network, such as a Bluetooth wireless network, in some embodiments a Bluetooth Low Energy (BLE) wireless network.

Additional description of the disclosed system, devices, and methods are provided below.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, e.g., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein.

In addition, components of each system and each process can be practiced independent and separate from other components and processes described herein. Each component and process can also be used in combination with other assembly packages and processes. The present aspects may enhance the functionality and functioning of computers and/or computer systems.

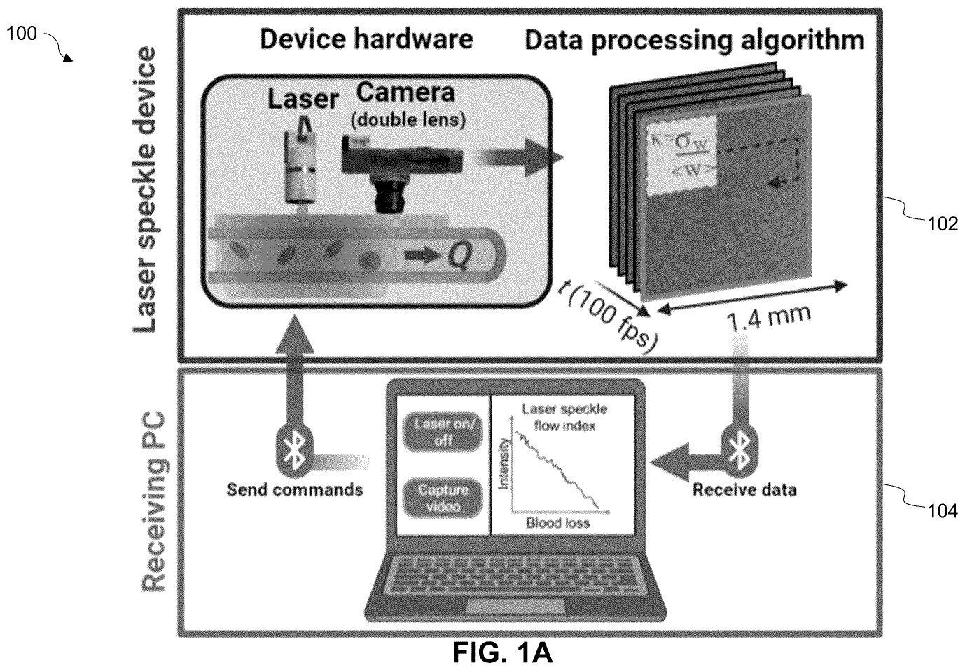

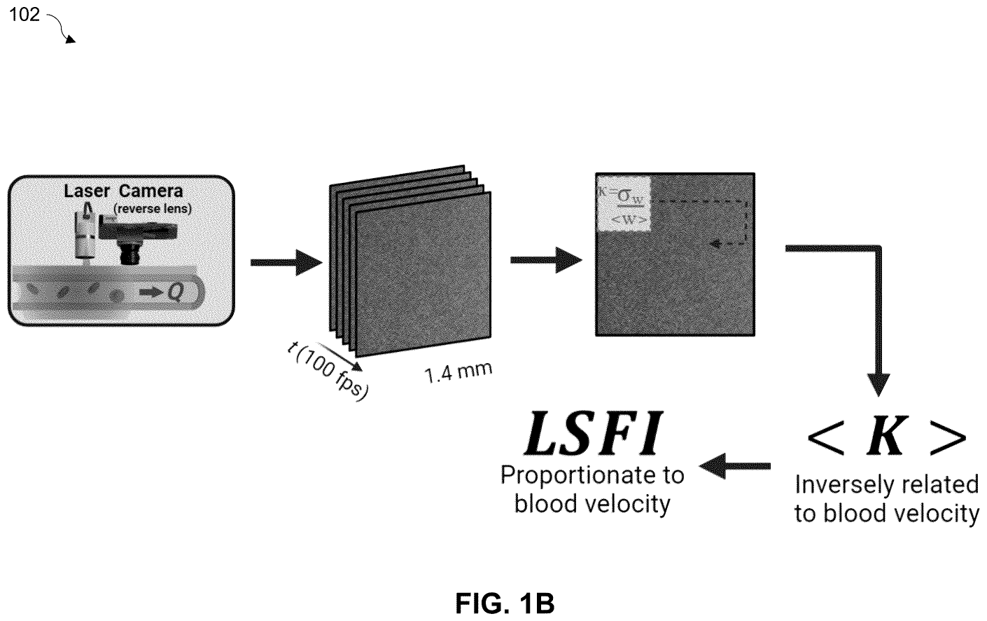

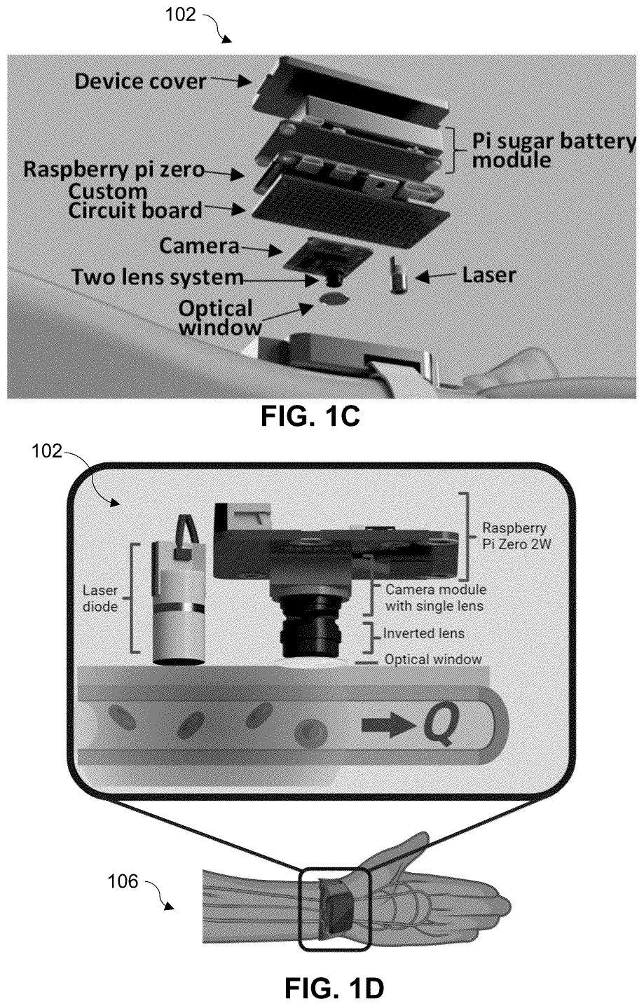

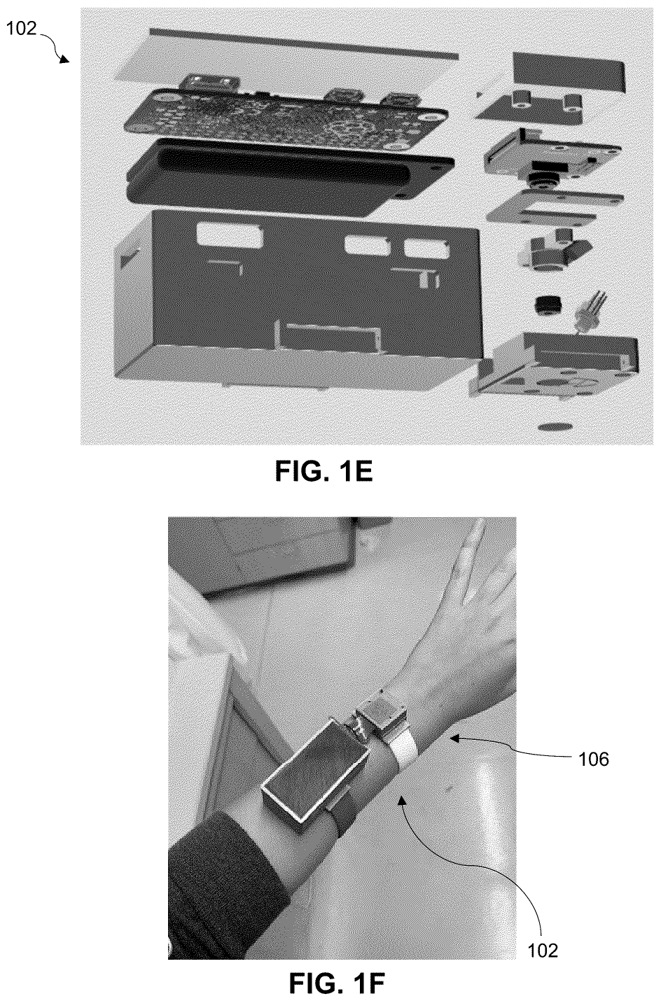

FIG. 1A illustrates a laser speckle imaging (LSI) system 100. LSI system 100 may include a laser speckle device 102 and a computing device 104 operatively connected to laser speckle device 102. Laser speckle device 102 may include at least a laser, a camera such as a double lens camera, a processor, and a memory in operative communication with the processor. The processor and memory may be configured to operate and/or control the laser and camera. The memory may have stored thereon a data processing algorithm capable of being executed by the processor. Computing device 104 may be configured as a workstation computer operatively connected to laser speckle device 102 and configured to (i) send commands to laser speckle device 102 such as to turn the laser on/off, and/or capture video via the camera, and (ii) receive data from laser speckle device 102. Computing device 104 may include a display and software for viewing data obtained from laser speckle device 102 and/or other results. The data processing algorithm may be rules-based and/or utilize artificial intelligence (AI) such as machine learning and models. The models may be trained, deployed, and updated (e.g., re-trained) over time with new data. The models may be image-based models capable of detecting objects and other features in relation to medical diagnostic images, and in particular in connection with hemodynamics aspects.

In some embodiments, laser speckle device 102 includes a low-cost laser diode and a Raspberry Pi camera and lens system (Raspberry Pi is a trademark of Raspberry Pi Ltd.). The controller of device 102 may be a Raspberry Pi zero which is used to process videos obtained by laser speckle device 102 and produce the LSFI signal. Laser speckle device 102 may also have wireless communication capabilities and be configured to send processed data to a computing device 104 for near real-time data processing and/or visualization. Additionally, computing device 104 may be configured to send commands to laser speckle device 102 to control laser function and video recording parameters.

In some embodiments, laser speckle device 102 includes a laser speckle sensor configured as an optical sensor designed in reflectance mode, e.g., the light source and detector are positioned on the same side, however a transmission mode design could also be used. The reflection mode device may include of a 50 mW, 780 nm laser module (e.g., from Laserland, 11071013) and a double-lens Raspberry Pi camera sensor. The double lens camera sensor may include a Raspberry Pi Camera Module V2, with the lens turned to have a maximal focal distance, and a second lens, from a second V2 module, inverted and attached directly to the surface of the first lens. A 0.4 mm sapphire window (e.g., from Edmund Optics, 43-628) is attached to the second lens, allowing for the sensor to be in focus on objects sitting or pressing directly on the window. The laser and double-lens camera sensor are held in position to be directly in contact with a subject by a 3D printed holder. A 3D printed holder that is adjustable and allows for the laser and sensor distance to be varied to a specified distance and then fixed may be used, for optimization of signal intensity and contrast. The laser module can be powered by a battery or a 3.3 V wall source. The camera sensor is powered and controlled by a Raspberry Pi 4 Model 4 computer board.

FIG. 1B illustrates additional aspects of laser speckle device 102. To monitor hemodynamics including situations such as hemorrhage (and/or postpartum hemorrhage), a laser speckle flow index (LSFI) can be derived from the inverse square of the speckle contrast index: such as LSFI=1 where <K> is an averaged speckle contrast index (described in more detail herein).

FIG. 1C illustrates an embodiment of one configuration laser speckle device 102. Laser speckle device 102 may include one or more housings, where each housing may include a cover and a body configured to be covered by the cover so that the housing is a closed/closable housing. Stored within one or more of the housings may be a plurality of components, including but not limited to a computer module, a laser (e.g., laser diode), a camera, a lens system, an optical window, a battery, and one or more additional circuit boards (e.g., custom circuit boards) configured, for example, provide power (e.g., a constant power output) for driving the laser. In some embodiments, the laser may be a 50 mW, 785 nm laser diode.

In some embodiments, the configuration in FIG. 1C may be battery-powered by a rechargeable Raspberry Pi sugar battery and include a stable current circuit for the laser diode. The camera may have a double lens system to allow the device to be in focus when in contact with an object. The configuration may be a low-cost (e.g., less than $100) and contain no consumables.

FIG. 1D illustrates a wearable embodiment of laser speckle device 102. Laser speckle device 102 may be configured with a strap or other securing implement to attach to a portion of a subject, such as subject 106. For example, in some embodiments, laser speckle device 102 may be configured for attachment to a wrist of a human subject.

FIG. 1E illustrates an alternative configuration of laser speckle device 102. The alternative configuration shown in FIG. 1E may use a different laser (e.g., laser) diode than the configuration shown in FIG. 1C, such as a smaller and more stable diode with lower optical power output up to 25 mW. We also split the device into two sections to reduce the bulk of the device.

FIG. 1F illustrates a real-world depiction of laser speckle device 102 mounted to a subject 106. Electronics withing housings of laser speckled device 102 may be operatively connected via a ribbon cable (other alternative configurations/designs of laser speckle device 102 may include all components integrated into one housing). Laser speckle images/videos obtained by laser speckle device 102 may be processed using one or more data processing algorithms as described herein to generate LSFI images/videos.

In some embodiments, the configuration of device 102 may be a wrist-watch form factor, battery powered (˜3-hour life), using Bluetooth data transfer, and providing near real-time data visualization and continuous monitoring, where the laser is a 780 nm laser diode.

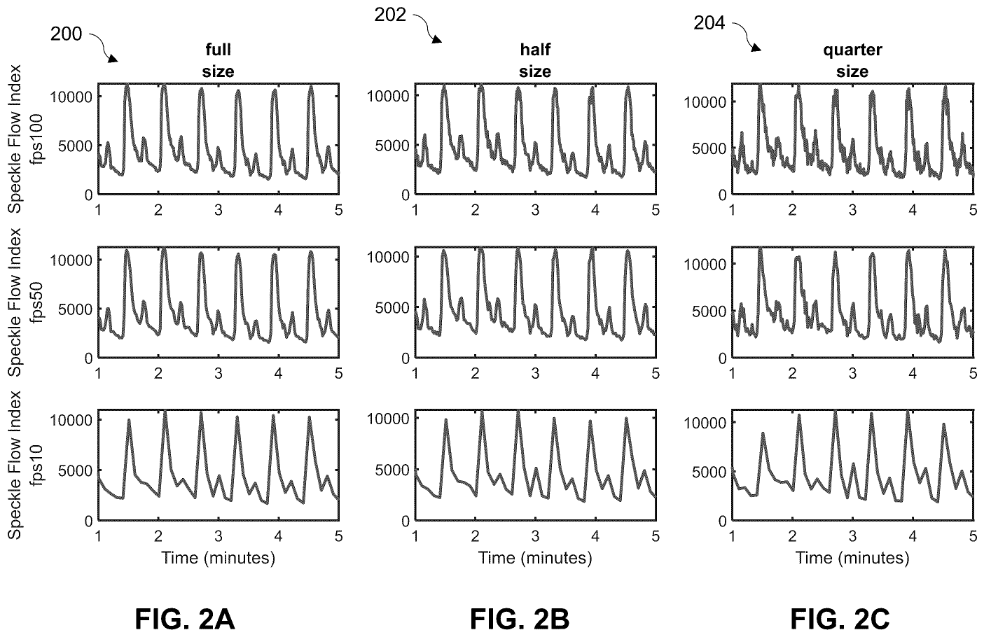

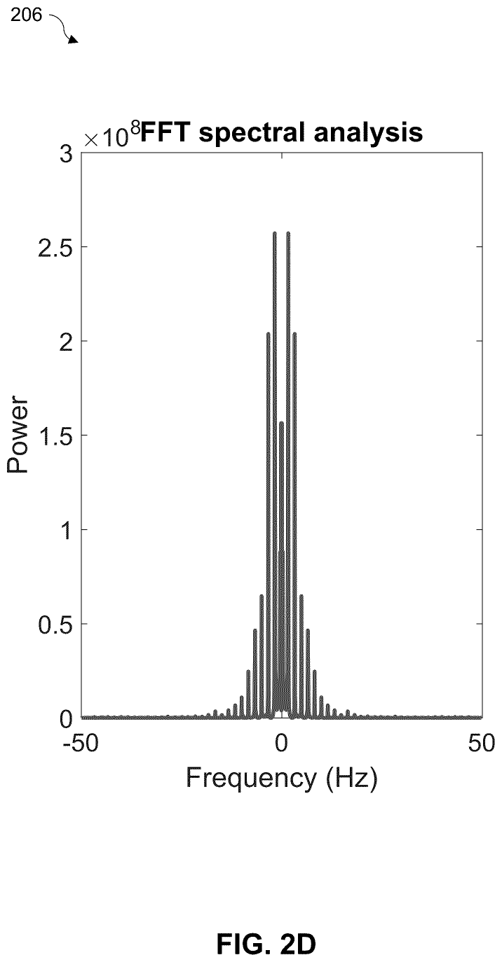

FIGS. 2A-2D illustrate SPG downsampling and FFT results obtained from a laser speckle device such as laser speckle device 102. FIGS. 2A, 2B, and 2C are plots illustrating various speckle flow indexes (e.g., fps 10, fps 50, fps 100) plotted against time. FIG. 2A illustrates plot 200 for full size, FIG. 2B illustrates plot 202 for half size, and FIG. 2C illustrates plot 204 for quarter size. FIG. 2D illustrates plot 206 for FFT spectral analysis.

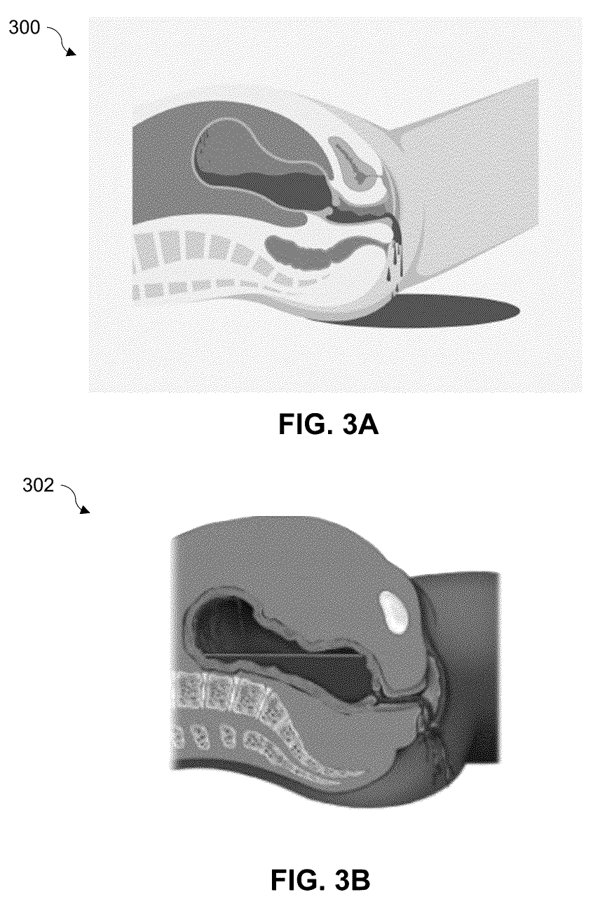

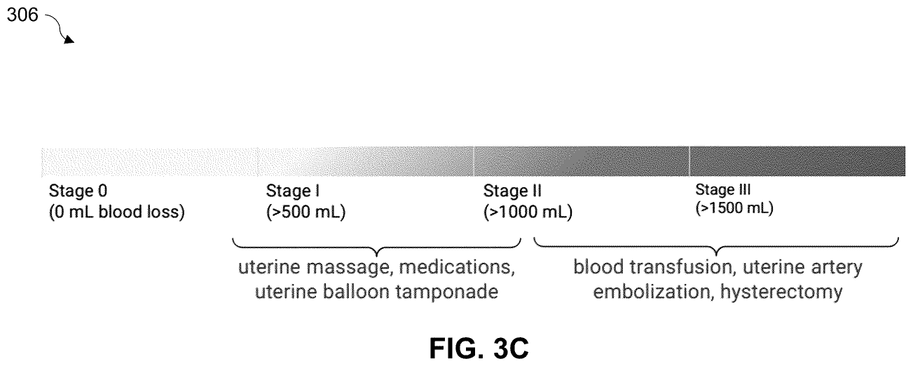

FIGS. 3A-3C illustrates aspects of PPH. FIG. 3A is a diagram 300 illustrating PPH in one subject. FIG. 3B is a diagram 302 illustrating PPH in another subject. Conventionally, blood loss estimation is performed using two main methods (i) visual: observe the quantity of blood-soaked sponges/dressings, and/or (ii) quantitative: volumetric collection mechanisms or weighing sponges/dressings, and vital sign monitoring includes monitoring blood pressure, heart rate, temperature spO2, respiratory rate, and/or Shock Index (=HR/BP). For visual blood loss estimation, medical personal will examine the amount of blood lost by the patients by viewing the quantity of blood-soaked sponges and dressings. This method can be improved by using volumetric blood collection technologies and weighing said sponges and dressings. The major limitation of this method is that internal bleeding will go undetected which results in underestimate of blood loss and PPH severity. Typically these methods are used in conjunction with vital sign monitoring, particularly shock index which is heart rate divided by blood pressure. This value increases with blood loss. However, vital sign changes are delayed by the bodies blood loss compensation mechanisms. Continuous monitoring of blood pressure is not plausible during delivery as arterial lines are not used in >99% of deliveries and typically a pneumatic cuff is used which takes about a minute to record a single data point. FIG. 3C is a diagram 304 illustrating stages (e.g., stages 0, I, II, III) of PPH.

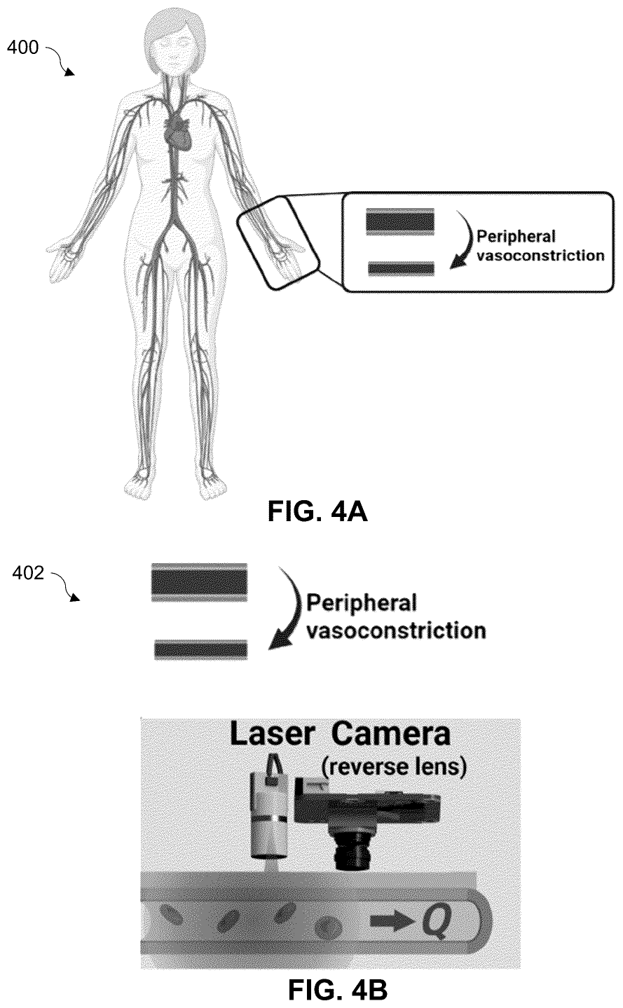

FIGS. 4A and 4B illustrate aspects of blood loss compensation. One dominant compensation mechanism is peripheral vasoconstriction. The body will shunt blood away from the periphery to redirect flow in a sense to vital organs and the body core (e.g., during hemorrhage the body will shunt blood from the periphery and maintain blood flow in the body core). Because of this, vital sign changes are delayed and early detection of PPH becomes challenging. This process delays vital sign changes as it stabilizes the body. FIG. 4A is a diagram 400 illustrating peripheral vasoconstriction. FIG. 4B is a diagram 402 illustrating peripheral vasoconstriction in connection with the laser and camera of a laser speckle device such as device 102. The disclosure herein recognizes solutions including (i) detecting blood loss during delivery and postpartum by monitoring the body's first line of defense: peripheral vasoconstriction, and (ii) measuring blood perfusion in the periphery using, for example, a reusable, low-cost, wearable laser speckle contrast imaging device as described herein.

PPH is a major problem in women's health and the outcomes of such are time dependent, meaning a diagnostic technology will need to provide an early indication of PPH. Additionally, this is a global health issue and thus requires a globally accessible solution that is low-cost, reusable, and preferably small and easy to use such as the device 102 described herein (e.g., by measuring blood velocity in the periphery with laser speckle contrast imaging).

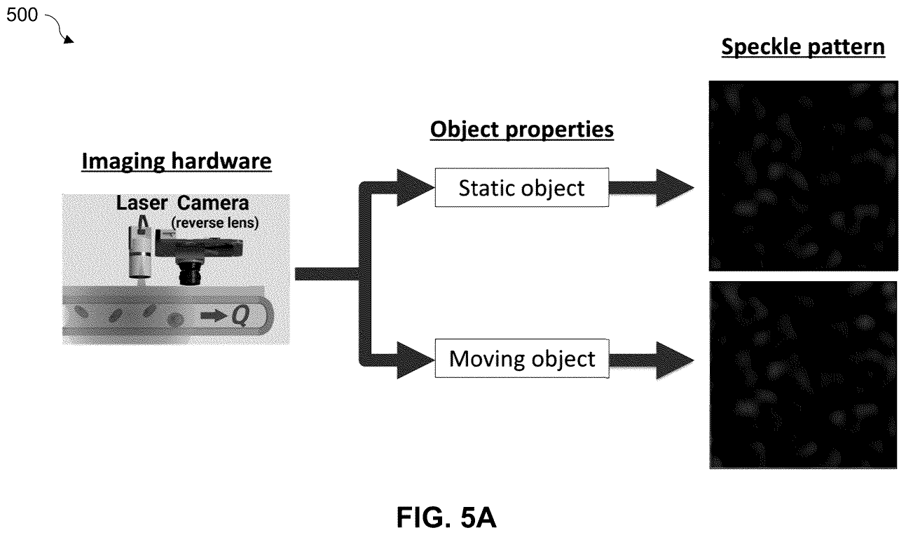

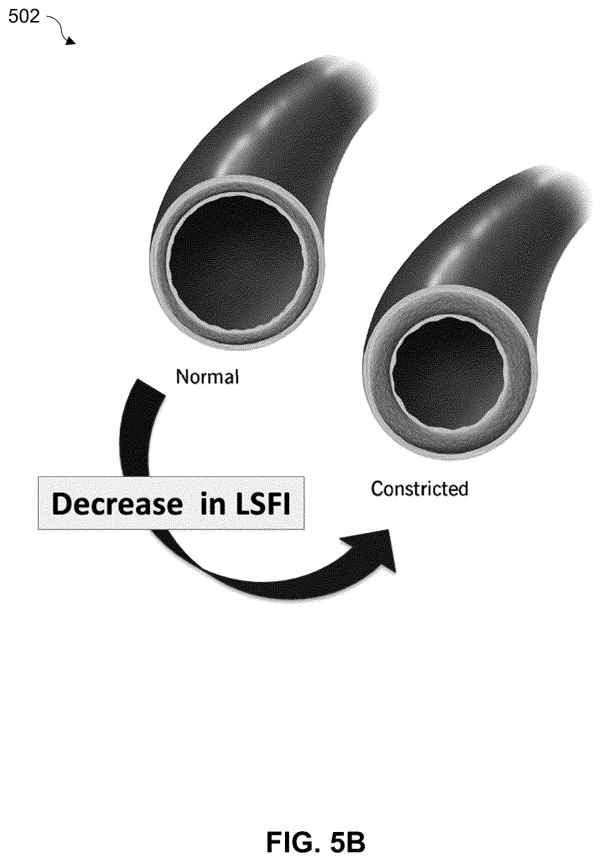

FIGS. 5A and 5B illustrate aspects of laser speckle contrast imaging. FIG. 5A is a diagram 500 illustrating laser speckle contrast imaging (LSCI), an imaging method that uses a laser light source and CMOS camera detector to quantify blood flow. This technology can be made small wearable and cheaply, making it amenable for use via device 102 described herein. Videos are captured of the random laser speckle pattern produced when laser light is interacts with tissue. This pattern changes dependent on tissue dynamics. An algorithm may be used to calculate the speckle contrast value or K. K is calculated by using a 7×7 pixel sliding window across a video frame and calculating the standard deviation over the mean pixel intensity for each window. An array of K values can be used to take the mean to get a single value per frame. The average K value can then be plugged into a laser speckle flow index to produce a single value per frame that is directly related to blood velocity. If this process is completed for every frame in a video, a time varying signal that is directly related to blood velocity results.

FIG. 5B is a diagram 502 illustrating aspects of LSCI and PPH detection. We can measure how quickly the speckles are moving using a well-documented algorithm to produce a signal called laser speckle flow index (LSFI). LSFI is a signal directly related to blood velocity. As a patient experiences blood loss via PPH, the peripheral vasoconstriction response will occur. Peripheral vasoconstriction should cause a decrease in blood flow and thus a decrease in LSFI signal in the periphery.

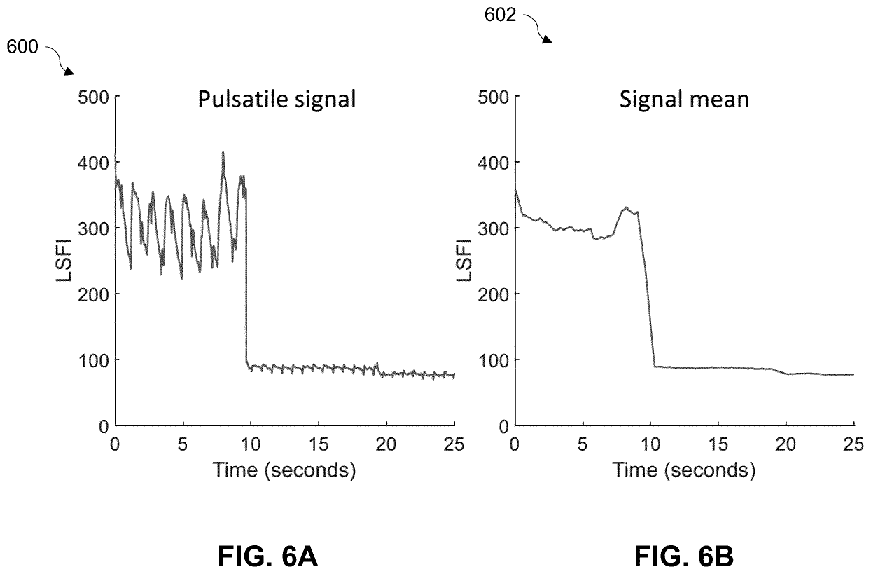

FIGS. 6A and 6B illustrate aspects of LSFI signals. FIG. 6A is a plot 600 illustrating pulsatile signal. FIG. 6B is a plot 602 illustrating signal mean.

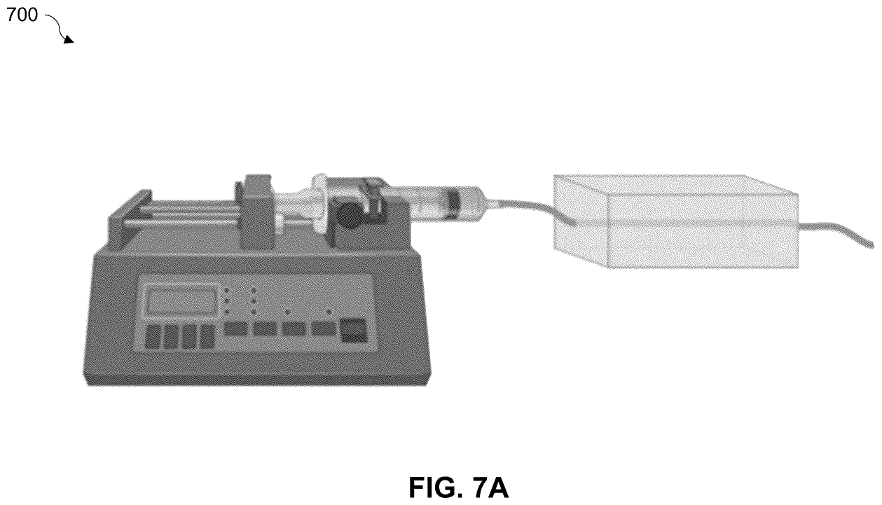

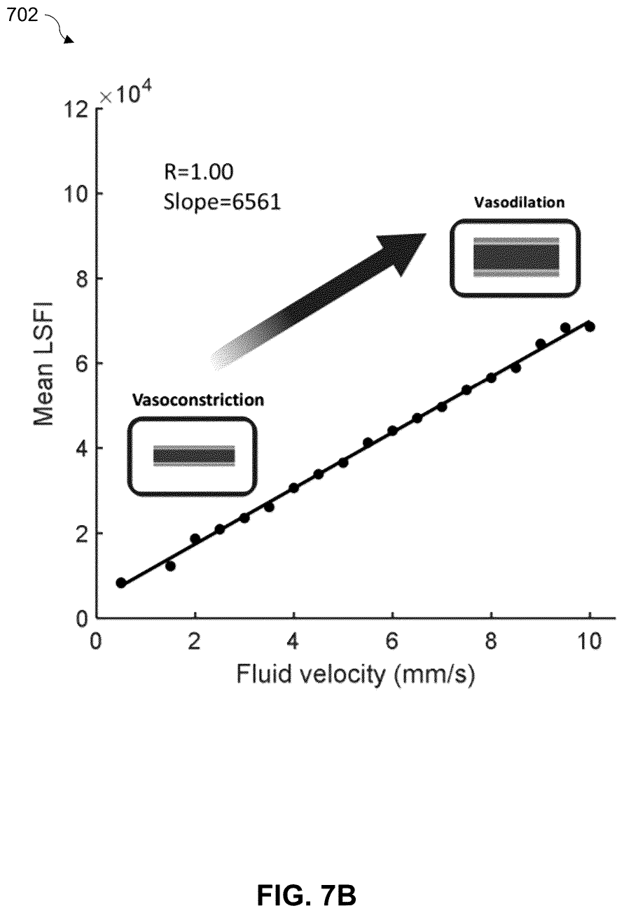

FIGS. 7A and 7B illustrate aspects of a flow phantom experimental setup. FIG. 7A illustrates a configuration 700 of an embodiment of a flow phantom experimental setup. A flow phantom procedure may include a 3D-printed optical flow phantom with a hollow channel, pumping fluid through the channel at physiologically relevant velocities (0-10 mm/s), and recording laser speckle video for a time such as 3 seconds at each velocity and find the mean LSFI. FIG. 7B illustrates diagram 702 of flow phantom results, showing linear correlation between LSFI signal and fluid velocity. A measured signal can be distinguished between different fluid velocities in high (vasodilated) and low (vasoconstricted) flow states (e.g., when mimicking periods of high and low flow velocity). In some embodiments, the correlation between LSFI and fluid velocity produced a correlation coefficient of 0.98. The performance of the device in vitro is comparable to high-cost benchtop systems.

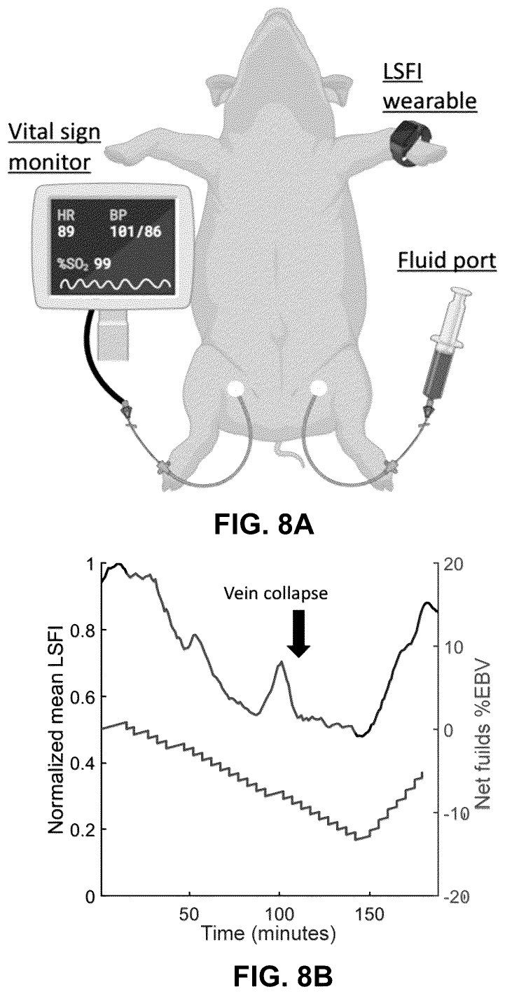

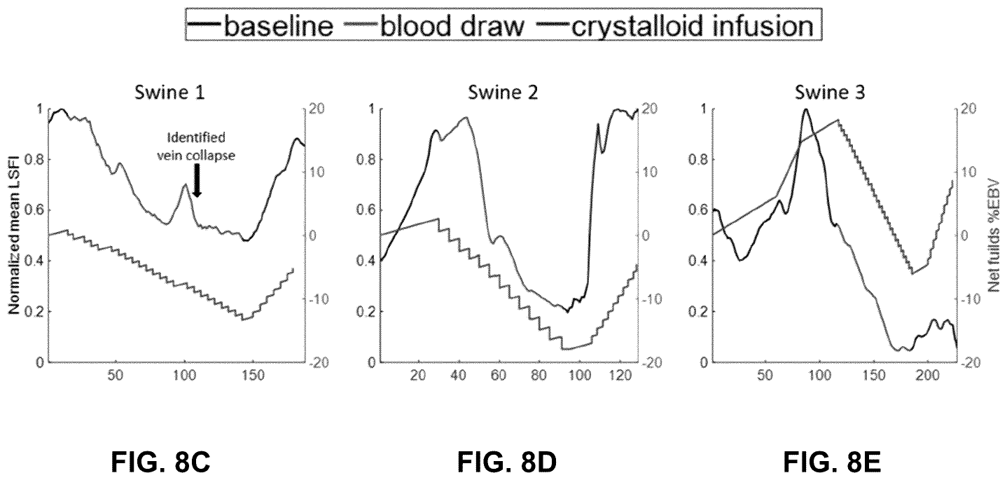

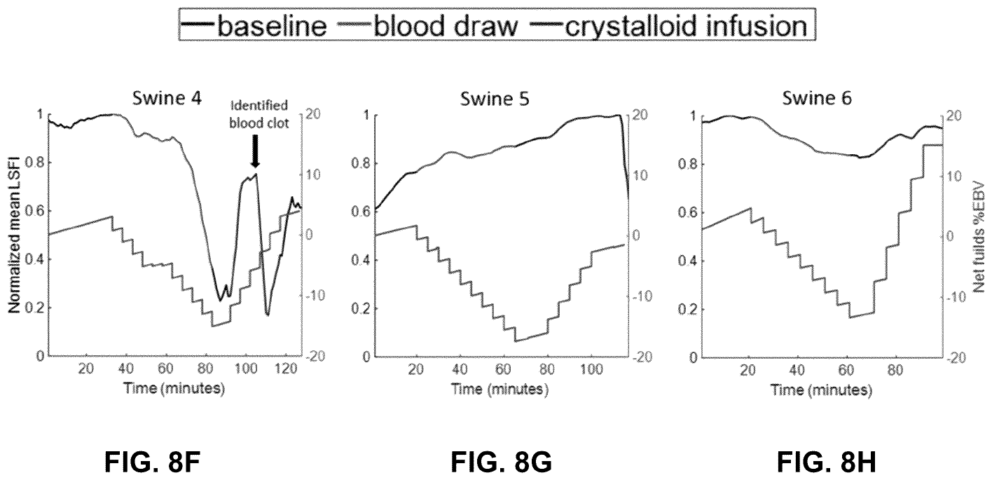

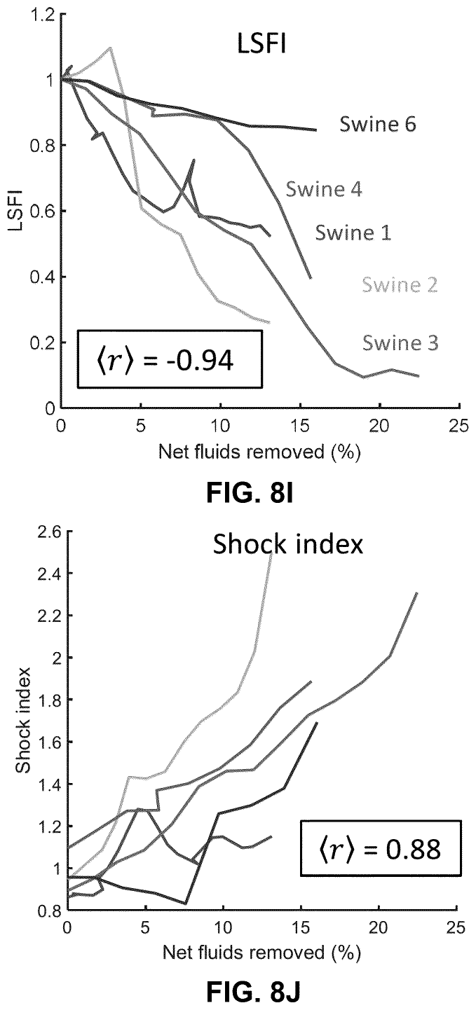

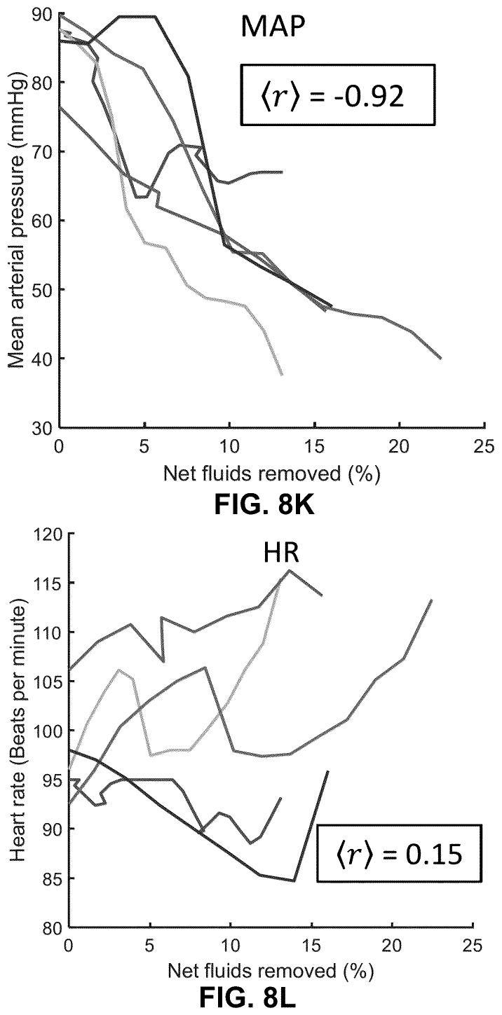

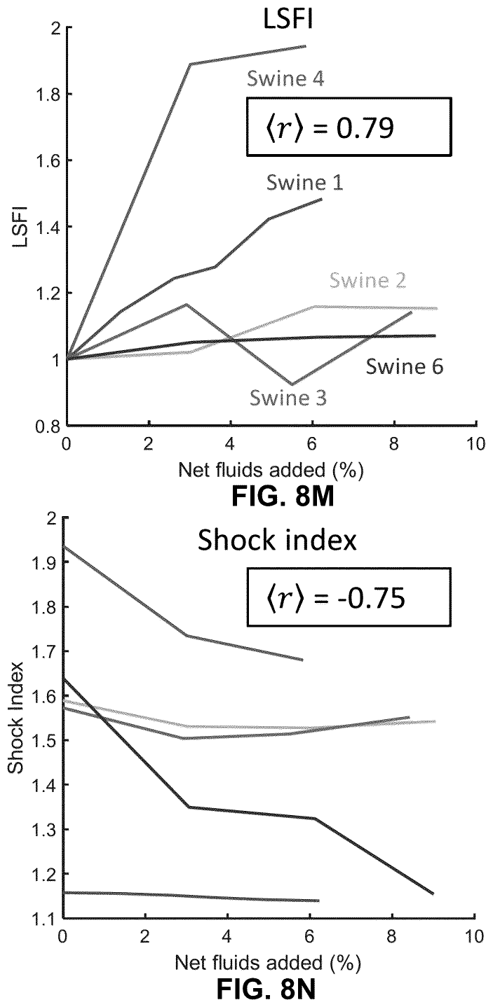

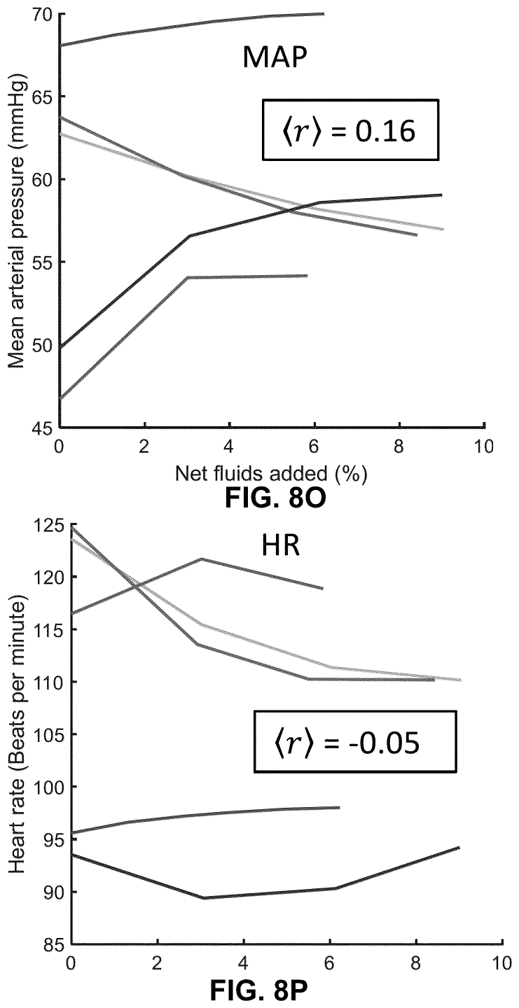

FIGS. 8A-8P illustrate aspects of a swine hemorrhage model. FIG. 8A illustrates a setup for testing a swine with a wearable such as device 102. Testing included: (1) Blood loss procedure: Up to 40% estimated blood volume (EBV) was removed at constant rate; (2) Crystalloid resuscitation procedure: Saline infused at a constant rate up to 20% of EBV; (3) Continuous vital sign monitoring: Heart rate, arterial blood pressure, temperature, respiratory rate, spO2. A swine hemorrhage model was used to quantify the blood loss detection ability of the device in vivo. Swine were anethsitized and the LSCI wearable was placed on the swine's left wrist. Data was recorded for 10 seconds every minute and the average LSFI was recorded for each minute. The blood loss procedure consisted of removing up to 40% estimated blood volume at a constant rate, which varied between swine. This was followed by a period of saline infusion of up to 20% estimated blood volume. Throughout the procedure vital signs such as heart rate and arterial blood pressure were monitored continuously.

FIG. 8B is a plot illustrating vein collapse. The green step function represents the net fluids into the swine over the procedure. The multicolored line represents the LSFI signal, with the red being the blood draw procedure, the blue being the saline infusion, and the black regions being baseline periods. Generally, a clear decrease in LSFI signal during blood loss and a clear increase during saline infusion observed (e.g., during blood loss LSFI signal decreases; during fluid infusion LSFI increases; vein collapse around 100 minutes appears as signal artifact.). Net fluids into the swine: add IV fluids and fluid infusions; remove blood.

FIGS. 8C-8H illustrates swine hemorrhage model results for 6 swine. The results include: (i) Swine #3: Did not recover from blood loss with fluid infusion. LSFI nearly reached zero. (ii) Swine #1/#4: Identified vascular events in LSFI signal. (iii) Swine #5: Received Heparin before study and did not appear to have a vasoconstriction response. Additionally, swine 3 LSFI signal essentially bottomed out at the end of blood loss and then never recovered much during the saline infusion, indicating there is a certain point of no return that may be identified. Additionally, the veterinary staff noticed a vein collapse during the blood draw procedure of swine 1 and a blood clot during the saline infusion procedure of swine 4, which we recorded as artifacts on the LSFI signal. This indicates likelihood of being able to detect certain vascular events.

FIGS. 8I-8L illustrate plots regarding quantitative analysis of blood loss. The plots display the mean correlation coefficient and slope for each signal. LSFI is normalized by the start of the blood loss procedure. The results of this study show a strong correlation between LSFI and net fluids of −0.94 especially considering that blood was drawn at different rates for each swine. This underscores the capability of the device 102 to monitor blood loss in vivo. Mean arterial pressure and shock index did correlate highly with net fluids as well, however these signals do not measure the same quantity as LSFI and they would likely all be useful to medical personnel. Additionally, during delivery mean arterial pressure and shock index cannot be measured continuously or in real-time, which LSFI can. LSFI produced the strongest correlation with blood loss. Shock index (SI) and MAP also correlated highly, but do not measure the same phenomena as LSFI. LSFI can be monitored continuously/close to real-time.

FIGS. 8M-8P illustrate plots regarding quantitative analysis of resuscitation. The analysis in FIGS. 8I-8L was repeated for just the resuscitation procedure. Again the correlation with LSFI produced the highest coefficient of 0.76. The correlation between LSFI and net fluids was lower for the resuscitation procedure compared to the blood loss procedure (e.g., likely because the resuscitation response is highly dependent on the degree on blood loss experienced by each swine and the individual physiological response). Here only shock index appears to be a relevant measure aside from LSFI, which mean arterial pressure and heart rate do not perform well. LSFI produced the strongest correlation with net fluids during resuscitation. Shock index (SI) also correlated highly, while MAP did not. In summary, the highest correlation between LSFI and net fluids for both the blood loss and resuscitation procedures compared to other metrics.

In some embodiments, a twelve week old male White Yorkshire x Landrace pig was anesthetized and cut downs were performed on the femoral vein and artery to establish a blood withdrawal port and to insert an arterial blood pressure catheter, respectively.

In some embodiments, data was collected using a Python script from the Raspberry Pi. Data was collected via video for 10 seconds every minute from 15 minutes before the start of the blood loss protocol, until 5 minutes after the final crystalloid infusion. Video data was saved directly onto the Raspberry Pi hard drive. Data was processed post-study as a rolling average of the speckle index over time. The camera was set to capture video at 100 frames per second, with a 5 ms exposure time.

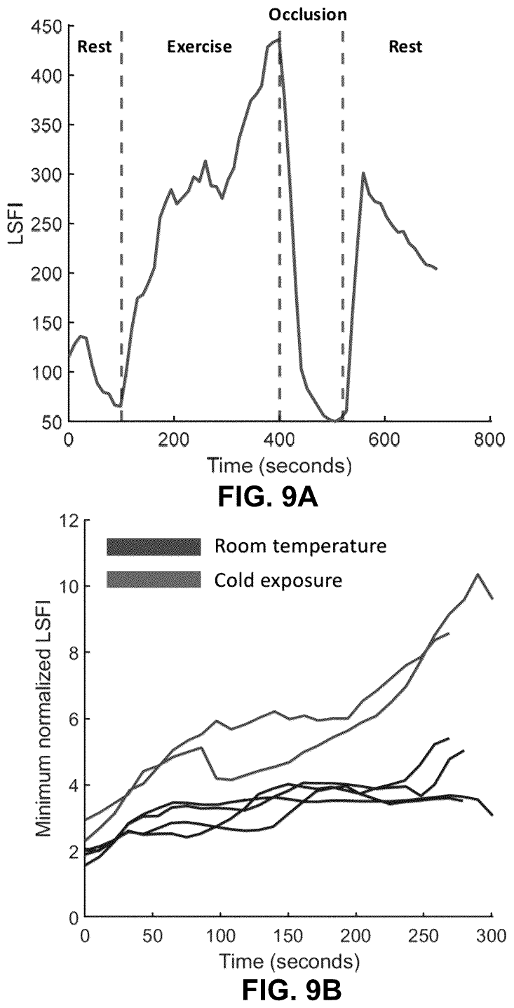

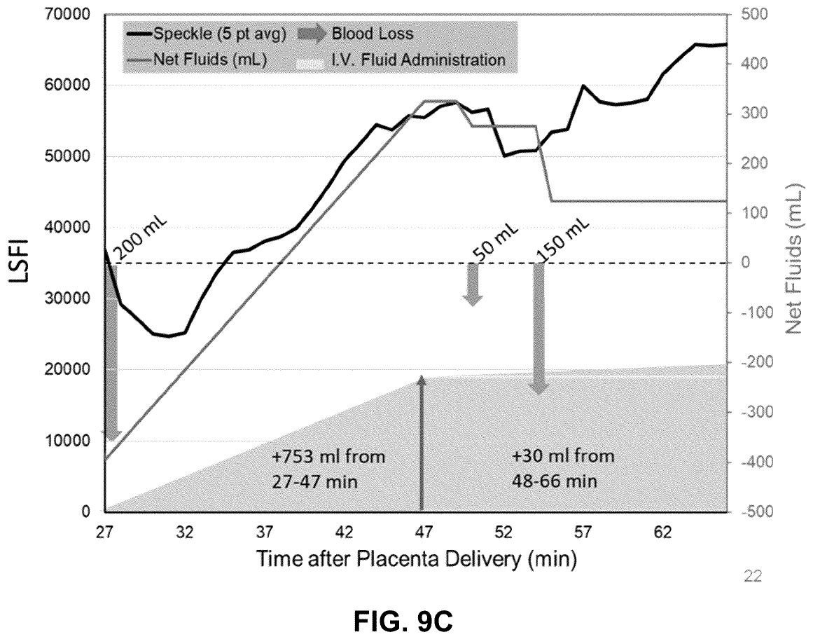

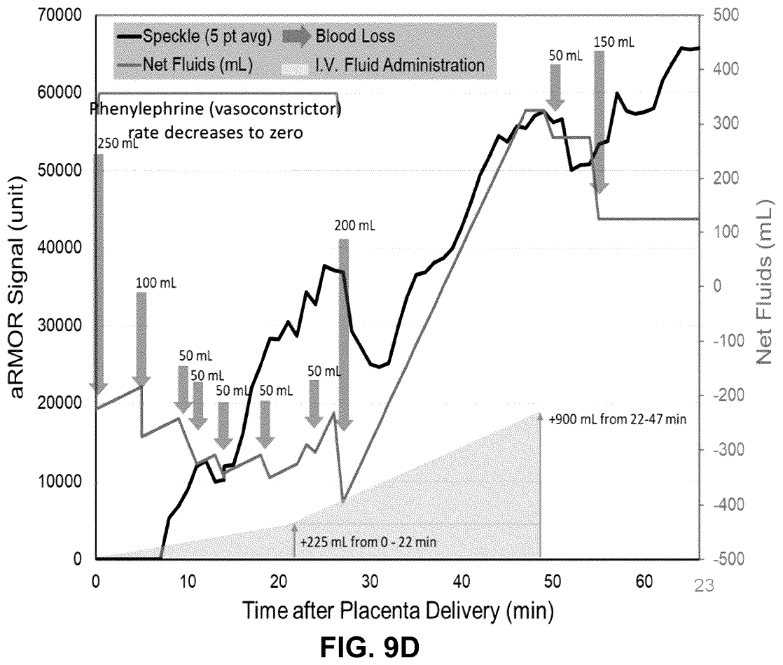

FIGS. 9A to 9D illustrate aspects of testing of healthy human volunteers. Volunteers undergo a series of physiological challenges to induce vasoconstriction and vasodilation responses, such as vasodilation caused by exercise on a recumbent stationary bike; and/or vasoconstriction caused through cold stimulation and arm occlusion. A comparison of results between non-pregnant and pregnant patients can be performed to characterize known hemodynamic adaptations that occur throughout pregnancy.

FIG. 9A—Heathy human volunteer protocol: 2 minutes of baseline recording; 5 minutes of exercise (vasodilation); 2 minutes of occlusion via pneumatic cuff (vasoconsticted/minimum flow); 3 minutes of rest. FIG. 9B—Two subject groups: (1) Just began with 5 minutes of exercise; (2) Placed their hand in cold water for 2-5 minutes prior to 5 minutes of exercise. Group 1: Mean rate of LSFI increase=0.021. Group 2: Mean rate of LSFI increase=0.007. Cold water prior to exercise caused a 3× reduction in the slope of the LSFI signal. FIG. 9C—Cesarean delivery results—stable medication levels (collected data from first pregnant patient, have to consider IV fluids given). FIG. 9D—Cesarean delivery results—dynamic medication levels (Have to consider the medications given (e.g., Phenylephrine, Oxytocin, Epidural). From this testing: a wireless low-cost wearable laser speckle contrast imaging device performed similarly to high-cost benchtop systems; LSFI signal repeatably correlates highly with net fluids during hemorrhage and treatment; able to identify vascular events and provide information about treatment response; device is capable of measuring vasoconstriction/dilation in human subjects. Additional implementations of device 102 and testing protocols may include: additional safety features; Wifi for wireless; accelerometer; real-time processing and display; patient-specific calibration; skin pigmentation characterization; physiology challenges with pregnant patients; restart recruitment of laboring and cesarean delivery patients; prioritizing vaginal to minimize interference from medications.

In some embodiments, to monitor hemorrhage (and/or postpartum hemorrhage), a laser speckle flow index (LSFI) can be derived from the inverse square of the speckle contrast index: LSFI=1 where <K> is an averaged speckle contrast index. This average can be defined different ways. The average speckle contrast <K> can be defined at a given time point as the average value across all pixels in a processed speckle video frame captured at that time point. One can also average across multiple frames, obtaining the average speckle contrast index over a 10 second time period, for example.

In some embodiments, in laser speckle contrast imaging, contrast is generated by applying a spatial averaging algorithm within a square sliding window that spans a raw speckle image. Specifically, to find the speckle contrast at a given pixel (x,y), one defines a square window centered about (x,y) and divides the standard deviation of the pixel intensity within that window by the mean pixel intensity within the window. For real time processing of video speckle data, this algorithm must be applied to every video frame, and with frame rates up to 100 fps, efficient speckle contrast algorithms are critical. The standard deviation of pixel intensity within each sliding window is related to the variance of the pixel intensity, which is determined by taking the difference between the mean of the square of the raw image pixel intensity and square of the mean of the raw image pixel intensity. An established approach for efficiently determining these rolling averaged images is convolving a square array of ones with both the square of the raw image and with the raw image itself, resulting in the following expression for the speckle contrast imagepixel intensity (k):where the sliding windows have dimensions n×n (n is odd without loss of generality), is the raw image intensity, and the ones matrices have the same dimension as the sliding window. The disclosed hemorrhage monitoring system captures a video stream, where each frame is a raw intensity image. To detect peripheral vascular flow, each frame is analyzed according to Equation 1 to yield a speckle contrast image k, and then the average pixel intensity <K> across the entire image (excluding a (n−1)/2 thick rectangular border) is calculated and stored for each frame. Thus, the output signal is a single averaged speckle contrast value over time. For each captured frame, the average speckle contrast <K> can be implemented in python using methods from publicly available software libraries like Numpy.mean( ), Numpy.ones( ) Scipy.signal.convolve2d( ), or Scipy.signal.fftconvolve( ). However, because the relevant output signal for monitoring is a measure of average speckle contrast and not the speckle contrast image itself, an alternative approach is possible that significantly speeds up processing time. To develop and validate a post-partem (PPH) hemorrhage monitoring system, the following experiments were conducted.

To develop and validate a PE and/or ED monitoring system, the following experiments were conducted.

As described herein, light-based wearables provide advantages over current diagnostic measures, including low cost, portable, non-invasive, little/no consumables, and no operator skill required.

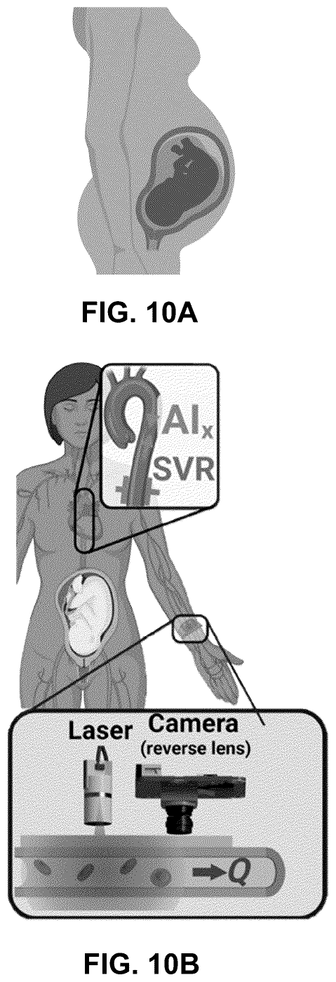

FIGS. 10A and 10B illustrate PE aspects. Cardiovascular features of PE include: (i) PE patients have increased arterial stiffness (e.g., measured via augmentation index (AIₓ)); (ii) Early-onset PE patients have higher systemic vascular resistance (SVR); (iii) PE patients have endothelial dysfunction. Non-invasively measure these items (i) to (iii) to provide a new diagnostic. AIₓ is the ratio of difference between systolic peak and inflection point to pulse pressure (P2-P1)/PP. AIₓ effectively measures the speed of the reflected systolic pressure wave, which returns faster in stiffer arteries and results in an increased AIₓ. Acronyms include: (i) Map: mean arterial pressure; (ii) RA: right arterial pressure/central venous pressure; (iii) CO: cardiac output; (iv) FMD: measures how much widening of artery when blood flows (artery does not bounce back).

### PE/ED Results

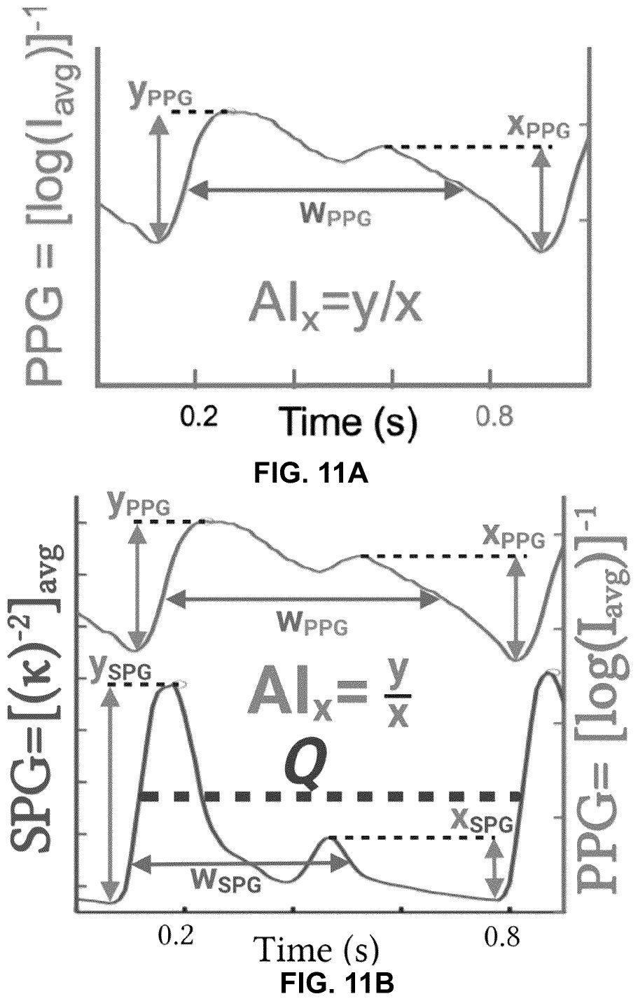

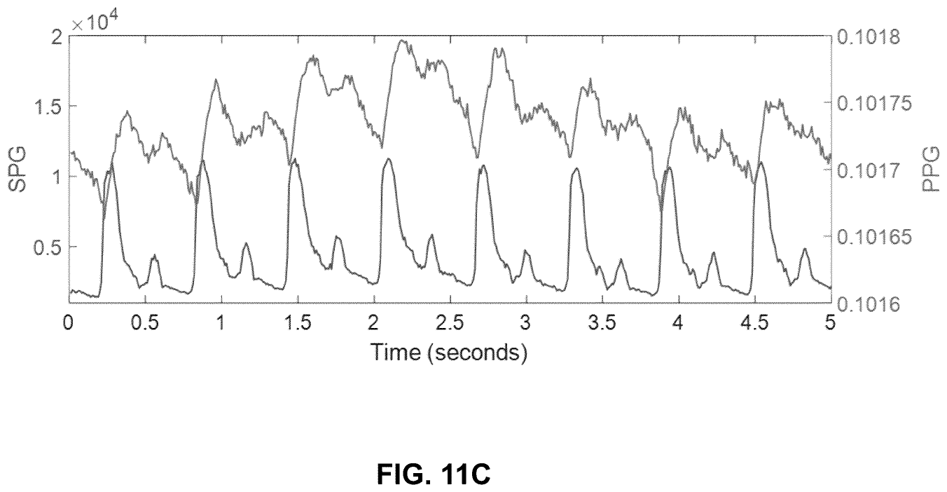

FIGS. 11A-11C illustrate aspects of light-based waveforms. FIG. 11A illustrates light-based cardiac cycle waveforms for providing diagnostic features (e.g., photoplethysmography). For example: PPG, senses changes in blood volume (BV). Limitations may include noisy waveforms reduce accuracy, low signal-to-noise ratio, measurements at extremely peripheral locations, issues with highly pigmented skin. FIGS. 11B and 11C illustrate how light-based SPG waveforms overcome limitations of PPG.

For a wearable device model for early detection of PE, device 102 may utilize an SPG sensor and derived SVR and AIₓ (compared to intra-arterial catheterization SVR and PPG-based AIₓ).

Research methods may include comparisons to gold standards.

Swine studies: with intra-arterial catheters and transthoracic echocardiography:

$$SVR = \frac{( M A P - C V P )}{C O}$$

AIx=y/x from PPG

Cardiac catheterization studies: Patients undergoing right and left heart catheterization

$$SVR = \frac{( M A P - R A )}{C O}$$

AIₓ=y/x from PPG

For each of the above studies, CVP=central venous pressure, MAP=mean arterial pressure, CO=cardiac output, RA=right arterial pressure

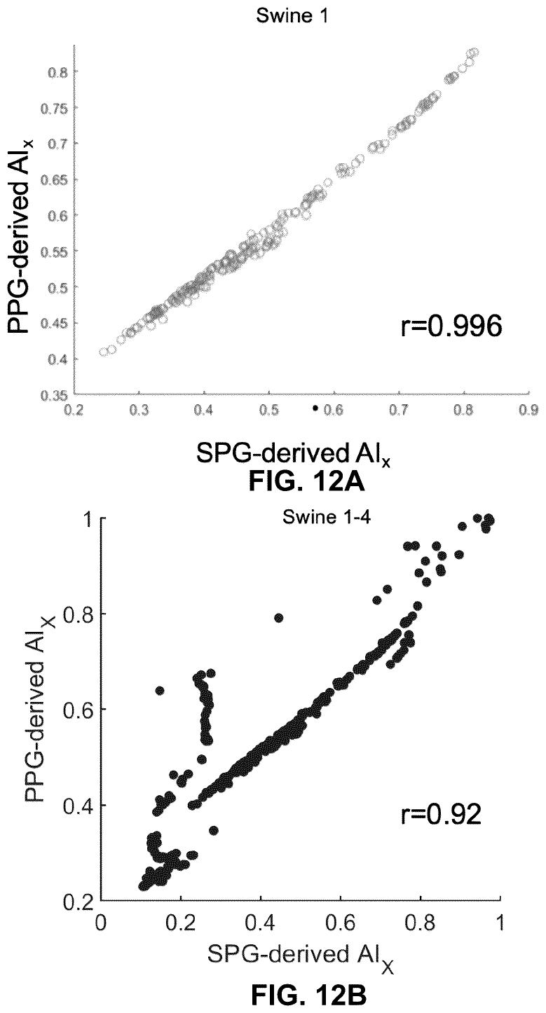

FIGS. 12A and 12B illustrate results from swine studies.

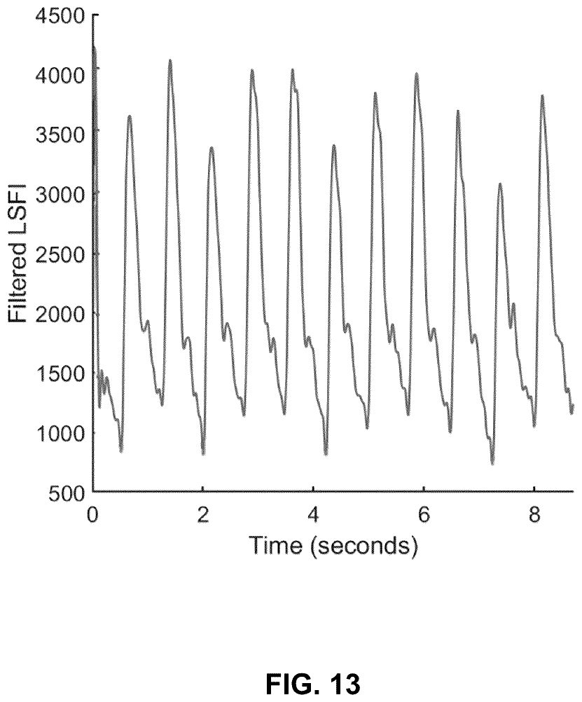

FIG. 13 illustrates results from cardiac catheterization lab.

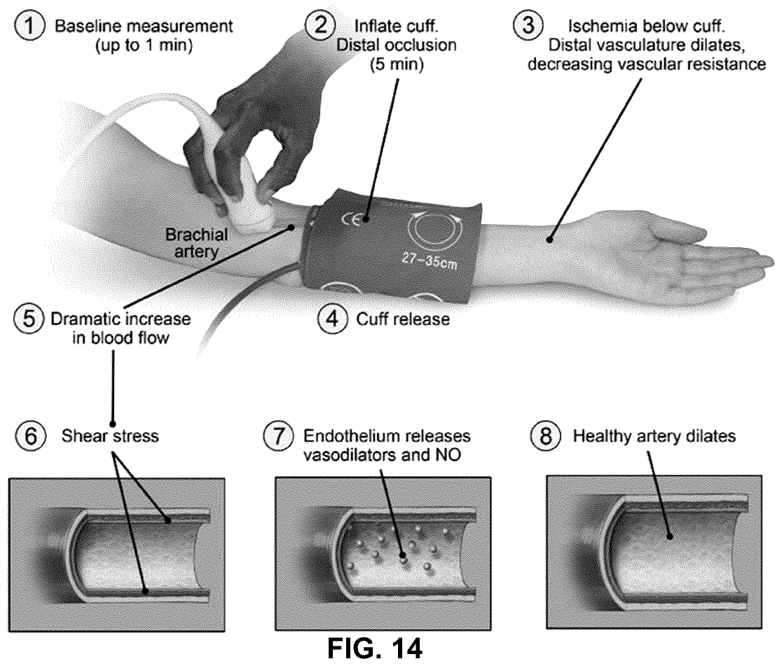

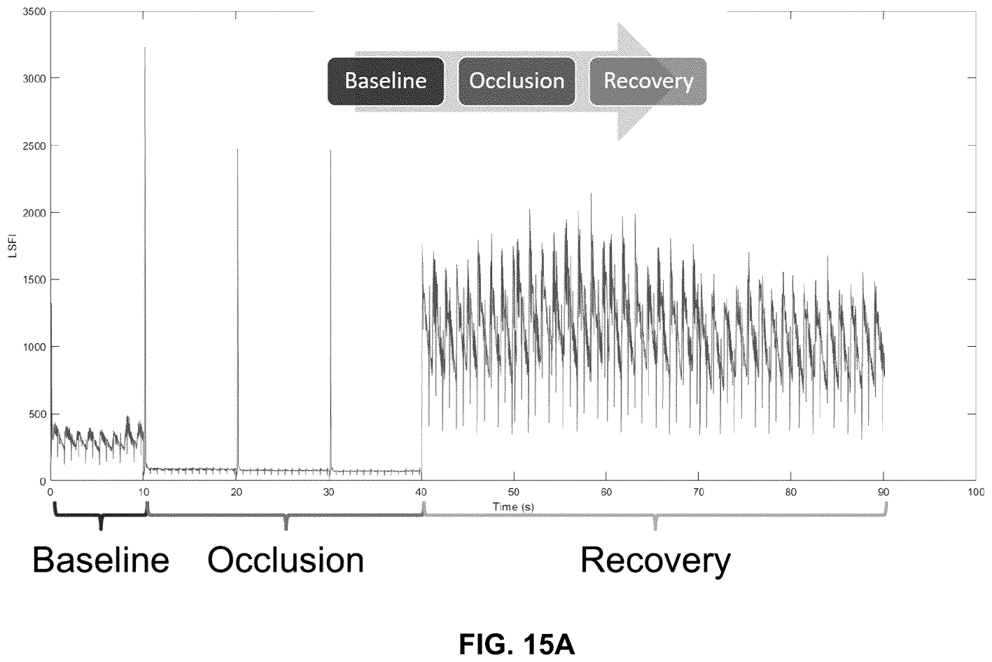

FIG. 14 illustrates light-based detection of ED. Flow mediated dilation (FMD) is the gold standard technique to measure endothelial dysfunction. Ultrasound probe measures diameter of brachial artery before, during, and after pneumatic cuff occlusion. During occlusion, vessels release nitric oxide (NO). Healthy vessels will dilate when exposed to NO. Healthy patients an increase in brachial artery diameter after occlusion. Limitations: expensive, requires expert operator, tedious, prone to error 15060-1871 (020809/US-NP)

For a wearable device model for early detection of PE/ED, device 102 may utilize an SPG sensor and derived FMD overshoot response (compared to ultrasound-derived FMD overshoot response).

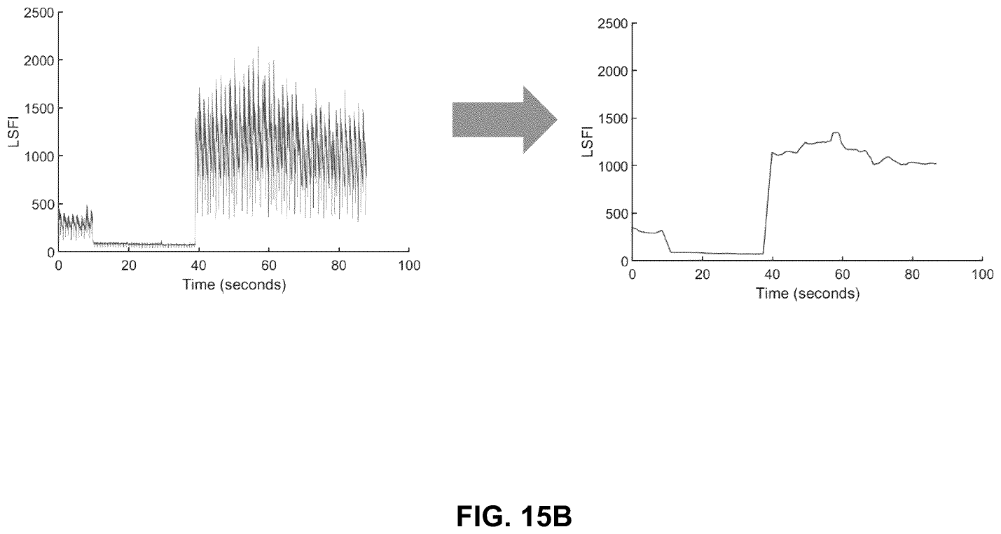

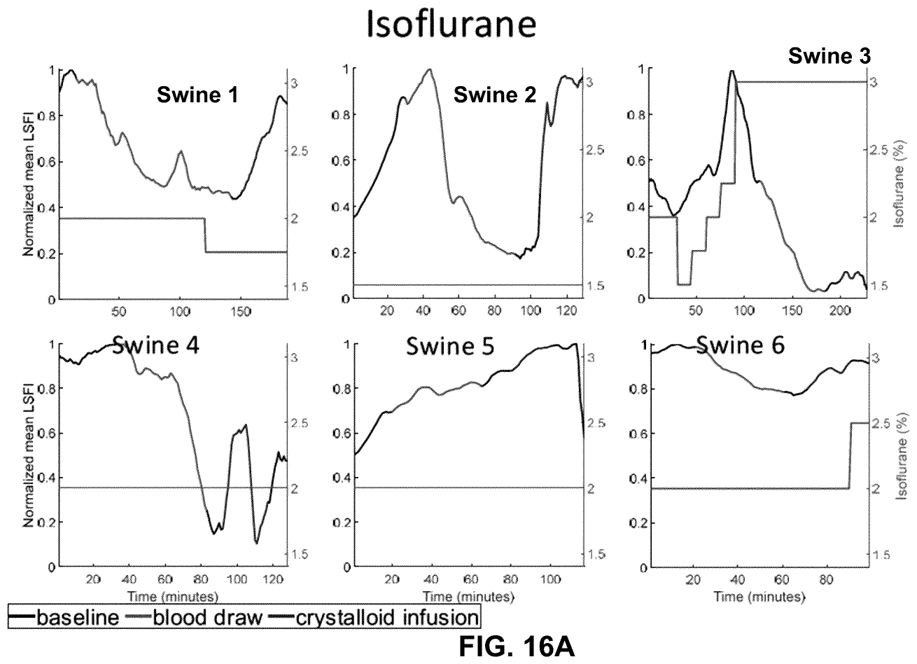

FIGS. 15A and 15B illustrate results of an embodiment of a research method for FMD comparisons to determine ED. Standard FMD tests include ultrasound brachial artery diameter. FIG. 15A illustrates FMD raw waveform. FIG. 15B illustrates FMD mean SPG waveform. Additional implementations may include: (i) Cardiac ICU— can get many SVR data points; (ii) with robust extraction and validation of SPG-derived SVR, AIₓ, and endothelial dysfunction—recruit pregnant patients; (iii) recruit pregnant patients with catheters and/or who are have echo Doppler performed.

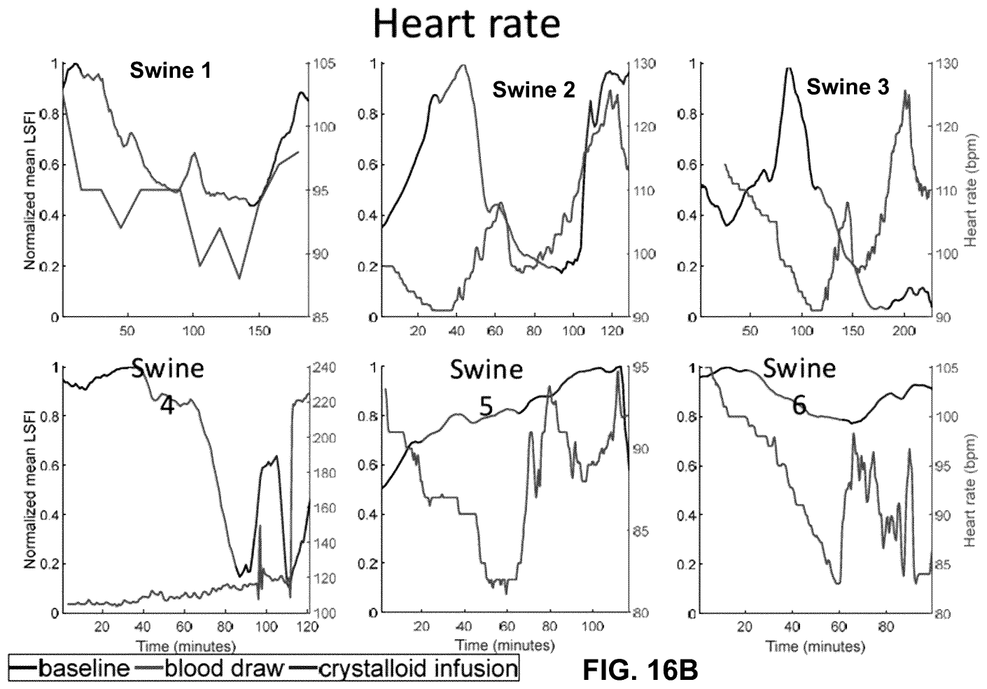

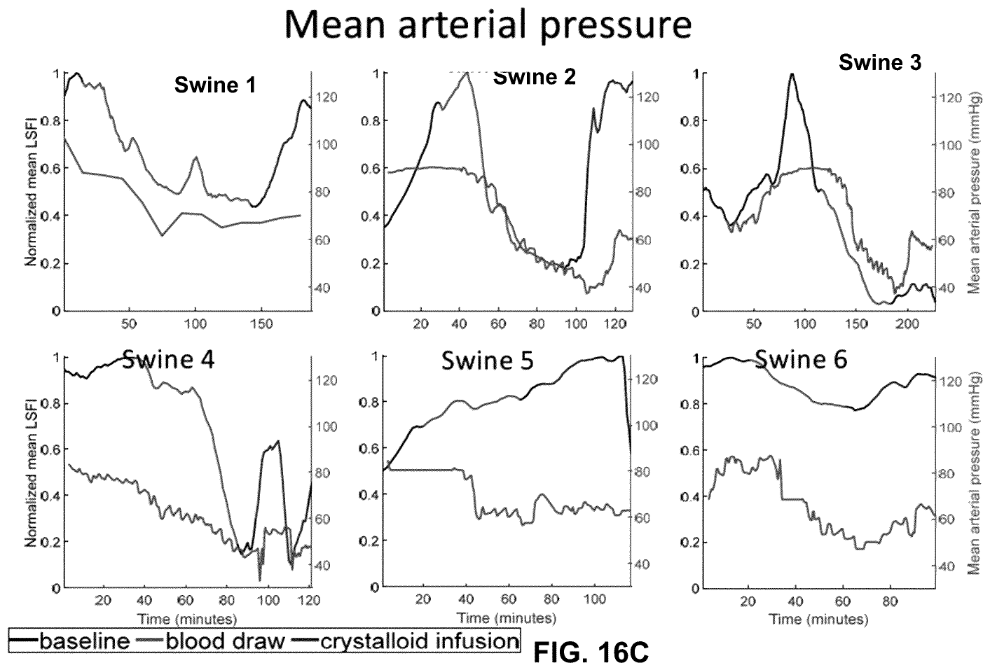

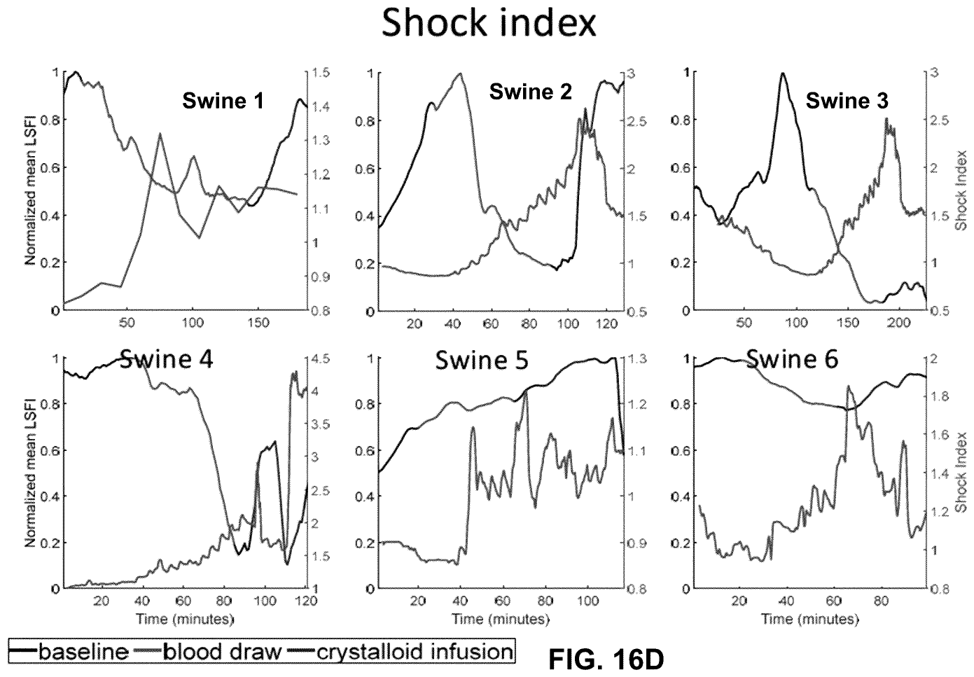

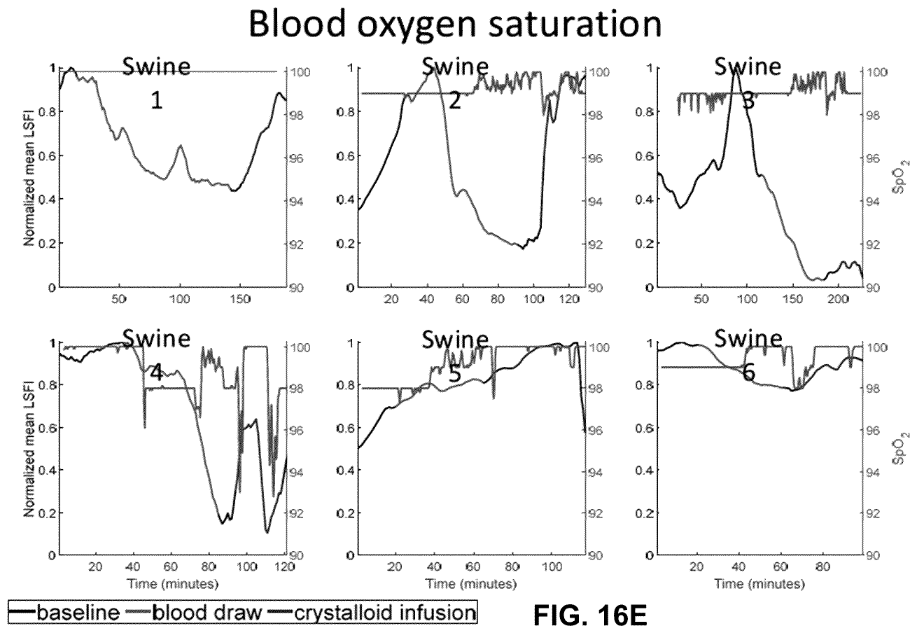

FIGS. 16A-16E illustrate additional swine test results.

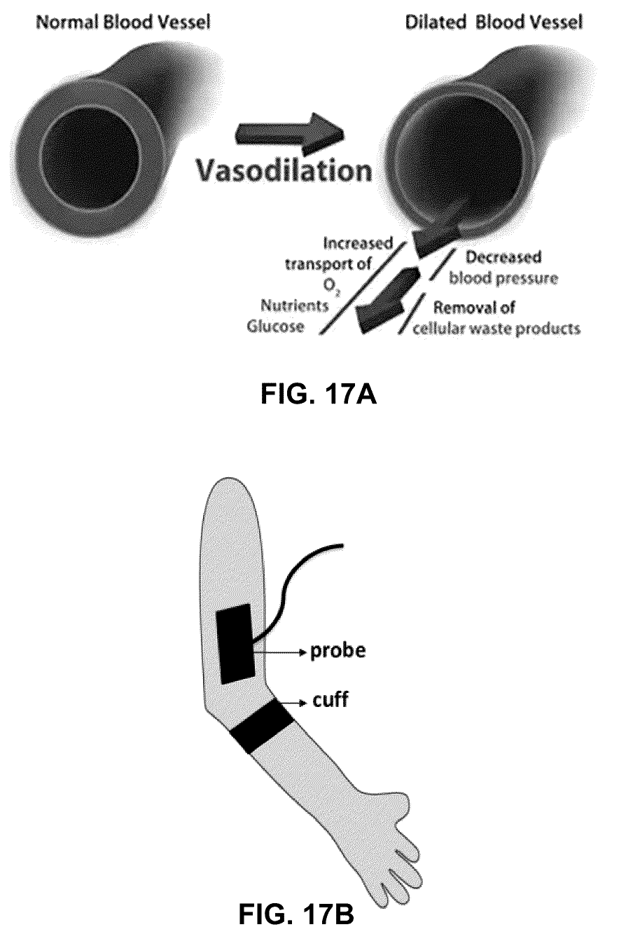

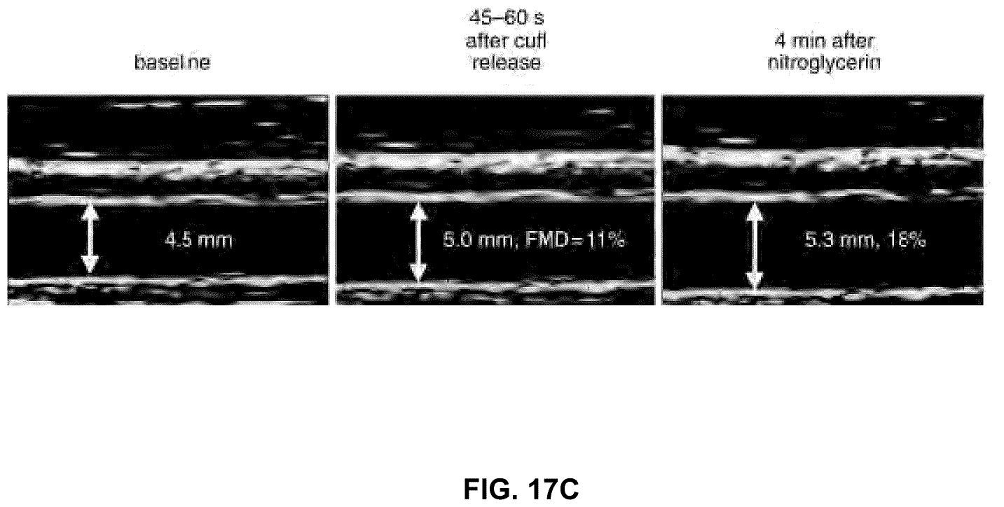

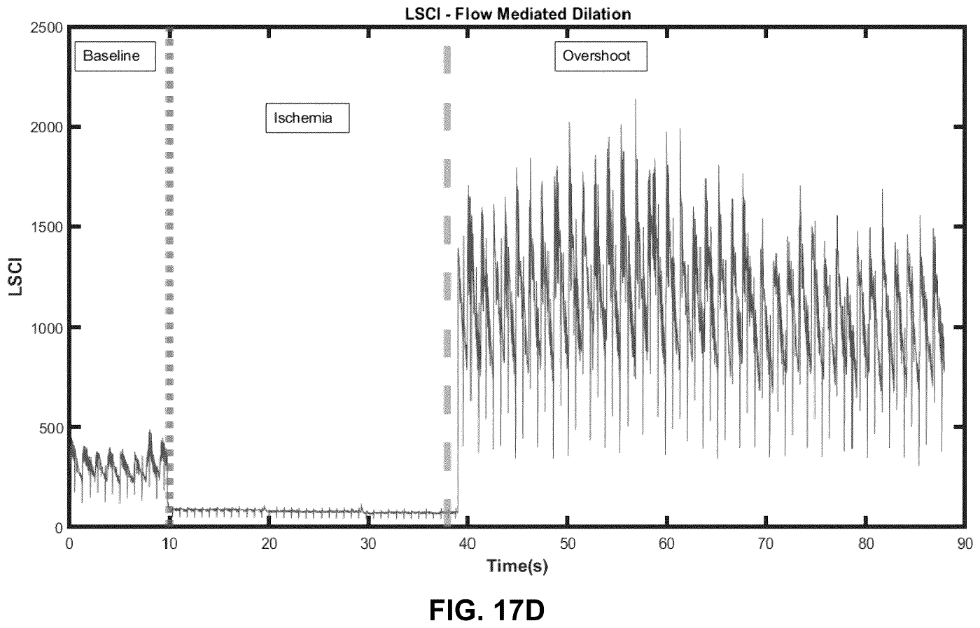

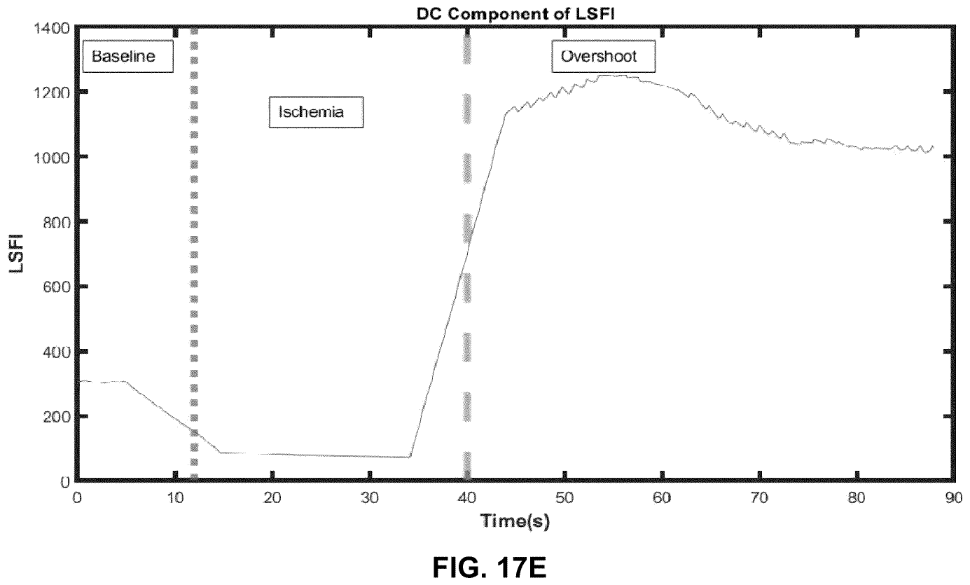

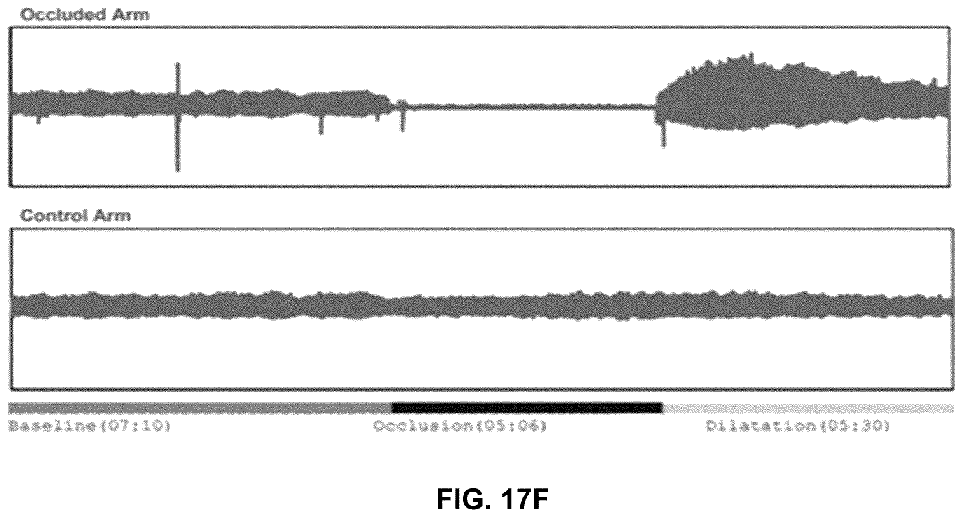

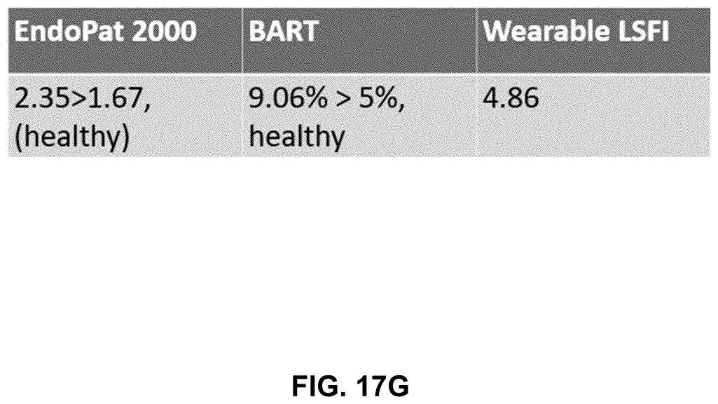

FIGS. 17A-17G illustrate additional comparisons and tests for detecting ED. FIG. 17A illustrates the vasodilation mechanism. FIG. 17B illustrates a BART/EndoPAT 2000 diagnostic setup. FIC. 17C illustrates results of the workflow of the FIG. 17B setup. FIG. 17D illustrates Raw LSFI signal during BART study—observed an increase in LSFI during ischemia recovery and decrease during ischemia. FIG. 17E illustrates mean LSFI signal during BART study. FIG. 17F illustrates EndoPAT2000 results during BART study. EndoPAT results confirm the overshoot response seen by wearable device 102 indicating healthy endothelial function:

$$RHI = \frac{post\_occlusion \_expt / pre\_occlusion \_expt}{post\_occlusion \_ctrl / pre\_occlusion \_ctrl}$$

FIG. 17G illustrates all three methods come to the same conclusion showing healthy endothelium. However, device 102 was shown to be more sensitive than previous methods. Device 102 is able to detect ischemia and hyperemia comparable to gold standard methods. Additional implementations include: more characterization of the RHI correlation between LSFI, BART and EndoPAT.

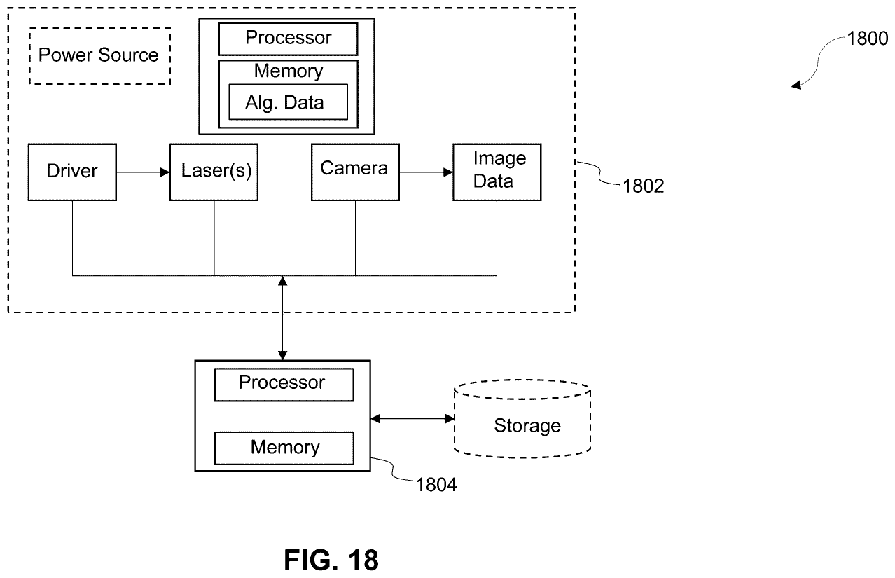

FIG. 18 illustrates a schematic 1800 of an embodiment of system 100 shown in FIG. 1A. A laser speckle device 1802 includes a processor, a memory storing algorithm data, a laser driver that drives a laser, and a camera that obtains images/videos, data of which is communicated to computing device 1804. In some embodiments device 1804 may be remote from device 1802 and interface with device 1820 via a network.

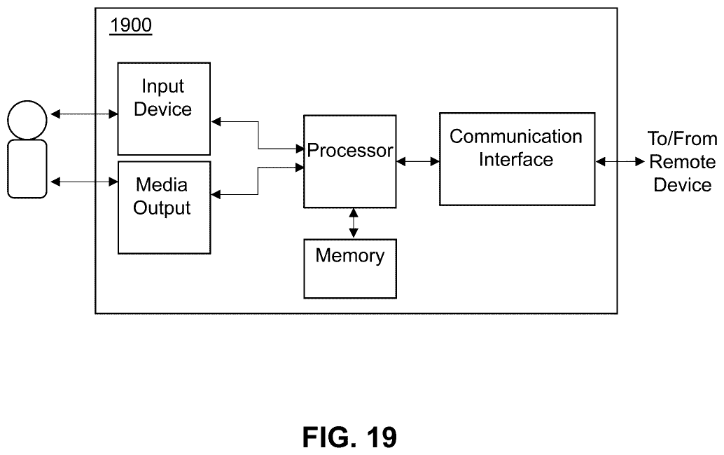

In another embodiment, device 104/1804 may be provided in the form of a computing device, such as a configuration 1900 of a computing device shown in FIG. 19. The computing device of configuration 1900 includes a processor for executing instructions. In some embodiments, executable instructions are stored in a memory area. Processor may include one or more processing units (e.g., in a multi-core configuration). Memory area is any device allowing information such as executable instructions and/or other data to be stored and retrieved. Memory area may include one or more non-transitory computer-readable media.

In another embodiment, the memory included in the computing device of configuration 1900 may include a plurality of modules. Each module may include instructions configured to execute using at least one processor. The instructions contained in the plurality of modules may implement at least part of the method for simultaneously regulating a plurality of process parameters as described herein when executed by the one or more processors of the computing device. Non-limiting examples of modules stored in the memory of the computing device include: a first module to receive data from a laser speckle device such as device 102/1802.

The computing device of configuration 1900 also includes one media output component for presenting information to a user. Media output component is any component capable of conveying information to a user. In some embodiments, media output component includes an output adapter such as a video adapter and/or an audio adapter. An output adapter is operatively coupled to processor and is further configured to be operatively coupled to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, the computing device of configuration 1900 includes an input device for receiving input from a user. Input device may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component and input device.

The computing device of configuration 1900 may also include a communication interface, which is configured to communicatively couple to a remote device such as server system or a web server. Communication interface may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)). The media output component may be configured to present information to a user, and input device may be configured to receive input from user.

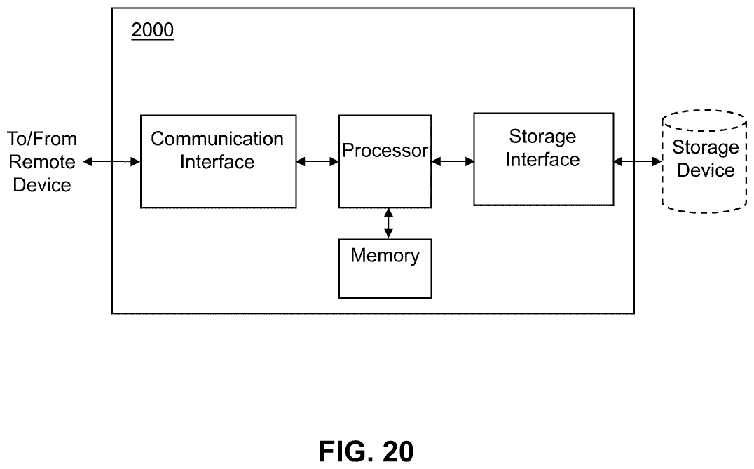

FIG. 20 illustrates an example configuration 2000 of laser speckle device 120/1802. In this aspect, the configuration 2000 includes a processor for executing instructions. Instructions may be stored in a memory area, for example. The processor may include one or more processing units (e.g., in a multi-core configuration) for executing instructions. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or any other suitable programming languages).

The processor is operatively coupled to a communication interface for communicating with a remote device, such as device 104/1804. For example, the communication interface may receive requests (e.g., requests to provide an interactive user interface to receive (e.g., sensor) inputs and to control one or more devices of system 100.

Processor may also be operatively coupled via a storage interface to a storage device, which may be cloud-based and accessible via a network Storage device is any computer-operated hardware suitable for storing and/or retrieving data. Storage device may be accessed by a plurality of server systems. Storage interface is any component capable of providing processor with access to storage device. Storage interface may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor with access to storage device.

Memory area may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), non-volatile RAM (NVRAM), registers, hard disk memory, a removable disk, a CD-ROM, or any other form of computer-readable storage medium known in the art. The above memory types are exemplary only, and are thus not limiting as to the types of memory usable for storage of a computer program.

Stored in memory are, for example, computer-readable instructions for providing a user interface to user via media output component and, optionally, receiving and processing input from input device. A user interface may include, among other possibilities, a web browser and an application. Web browsers enable users to display and interact with media and other information typically embedded on a web page or a website from a web server. An application allows users to interact with a server application.

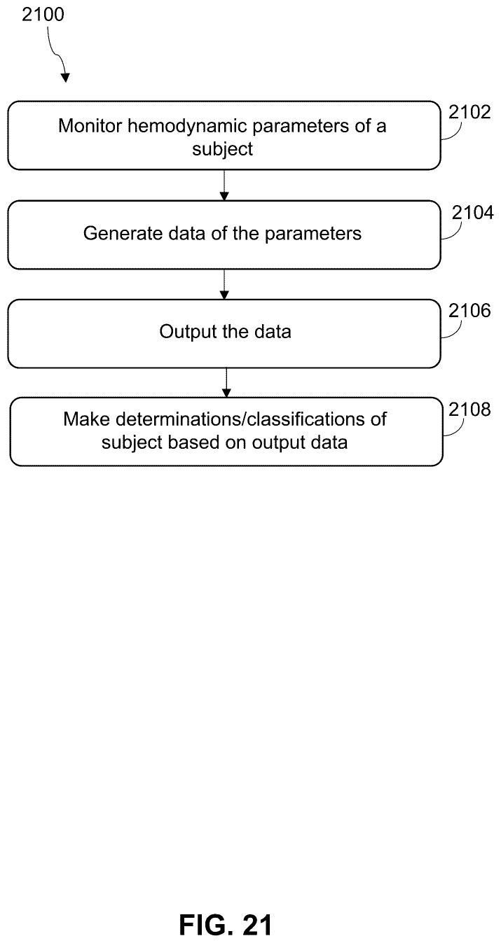

FIG. 21 illustrates an example process flow according to one embodiment of the present disclosure. Method 2100 includes monitoring 2102 hemodynamic parameters of a subject, such as for PPH, PE/ED as described herein. Method 2100 includes generating 2104 data corresponding to the parameters. This may include image/video data as described herein. Method 2100 includes outputting 2104 the data, for example to computing device 104/1804 as described herein. Method 2100 includes making 2106 determinations/classifications of the subject based on the data. This may include device 104/1804 analyzing the data to provide real-time detections/alerts as described herein. Alternatively, making 2106 determinations/classifications may be performed by device 102/1820 itself, thereby providing real-time detection/alert capability for PPH and PE/ED as described herein.

Although the present disclosure is described in connection with an exemplary imaging system environment, embodiments of the invention are operational with numerous other general purpose or special purpose imaging system environments or configurations. The imaging system environment is not intended to suggest any limitation as to the scope of use or functionality of any aspect of the invention. Moreover, the imaging system environment should not be interpreted as having any dependency or requirement relating to any one or combination of components illustrated in the exemplary operating environment. Examples of well-known imaging systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Computer systems, as described herein, refer to any known computing device and computer system. As described herein, all such computer systems include a processor and a memory. However, any processor in a computer system referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel. Each of the memories described herein may be non-transitory storage mediums.

The term “processor,” as used herein, refers to central processing units, microprocessors, microcontrollers, reduced instruction set circuits (RISC), application specific integrated circuits (ASIC), logic circuits, and any other circuit or processor capable of executing the functions described herein. The above are examples only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In one embodiment, a computer program is provided to enable the data processing of the MRI method as described herein above, and this program is embodied on a computer readable medium. In an example embodiment, the computer system is executed on a single computer system, without requiring a connection to a server computer. In a further embodiment, the computer system is run in a Windows® environment (Windows is a registered trademark of Microsoft Corporation, Redmond, Washington). In yet another embodiment, the computer system is run on a mainframe environment and a UNIX® server environment (UNIX is a registered trademark of X/Open Company Limited located in Reading, Berkshire, United Kingdom). Alternatively, the computer system is run in any suitable operating system environment. The computer program is flexible and designed to run in various different environments without compromising any major functionality. In some embodiments, the computer system includes multiple components distributed among a plurality of computing devices. One or more components may be in the form of computer-executable instructions embodied in a computer-readable medium.

The computer systems and processes are not limited to the specific embodiments described herein. In addition, components of each computer system and each process can be practiced independent and separate from other components and processes described herein. Each component and process also can be used in combination with other assembly packages and processes.

Exemplary embodiments of methods, systems, and apparatus for use in frame-wise multi-echo distortion correction for fMRI are described above in detail. The methods, systems, and apparatus are not limited to the specific embodiments described herein but, rather, operations of the methods and/or components of the systems and/or apparatus may be utilized independently and separately from other operations and/or components described herein. Further, the described operations and/or components may also be defined in, or used in combination with, other systems, methods, and/or apparatus, and are not limited to practice with only the systems, methods, and apparatus described herein.

The order of execution or performance of the operations in the embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

It will be understood by those of skill in the art that information and signals may be represented using any of a variety of different technologies and techniques. For example, data, instructions, commands, information, signals, bits, symbols, and/or chips may be represented by voltages, currents, electromagnetic waves, magnetic fields or particles, optical fields or particles, or any combination thereof. Similarly, the various illustrative logical blocks, modules, circuits, and algorithm operations described herein may be implemented as electronic hardware, computer software, or a combination of both, depending on the application and the functionality. Moreover, the various logical blocks, modules, and circuits described herein may be implemented or performed with a general purpose computer, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. Exemplary general purpose processors include, but are not limited to only including, microprocessors, conventional processors, controllers, microcontrollers, state machines, or a combination of computing devices.

When introducing elements of aspects of the invention or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computing device for monitoring hemodynamic status, the computer device including at least one processor in communication with at least one memory device, a light source, and an imaging device, the at least one processor programmed to:
operate the light source to emit light toward a portion of a subject;
obtain subject data via the imaging device, the subject data including at least hemodynamic parameters of the subject;
analyze the subject data to determine if a parameter of the hemodynamic parameters is indicative of a health issue relating to blood flow; and
generate an output regarding the health issue, the output configured to apprise a user of the health issue.

2. A computer-implemented method for monitoring hemodynamic status, the method implemented by a computer device including at least one processor in communication with at least one memory device, a light source, and an imaging device, the method comprising:
operating the light source to emit light toward a portion of a subject;
obtaining subject data via the imaging device, the subject data including at least hemodynamic parameters of the subject;
analyzing the subject data to determine if a parameter of the hemodynamic parameters is indicative of a health issue relating to blood flow; and
generating an output regarding the health issue, the output configured to apprise a user of the health issue.

3. One or more non-transitory computer-readable storage media for a computing device providing monitoring of hemodynamic status, the one or more non-transitory computer-readable storage media comprising a plurality of instructions stored thereon that, in response to being executed, cause the computing device to:
operate the light source to emit light toward a portion of a subject;
obtain subject data via the imaging device, the subject data including at least hemodynamic parameters of the subject;
analyze the subject data to determine if a parameter of the hemodynamic parameters is indicative of a health issue relating to blood flow; and
generate an output regarding the health issue, the output configured to apprise a user of the health issue.
